(19) United States
(12) Patent Application Publication
(10) Pub. No.: US 2012/0219538 A1
(43) Pub. Date: Aug. 30, 2012

(54) STABILIZED PROTEIN FORMULATIONS AND USE THEREOF

(75) Inventors: Gerrit Borchard, Arzier (CH); Claudia Mueller, Geneve (CH); Martinus Anne Hobbe Capelle, Oberwil (CH); Tudor Arvinte, Riehen (CH)

(73) Assignees: THERAPEOMIC AG, Riehen (CH); UNIVERSITE DE GENEVE, Geneva 4 (CH)

(21) Appl. No.: 13/505,491

(22) PCT Filed: Nov. 1, 2010
(86) PCT No.: PCT/IB10/54927

§ 371 Date: May 2, 2012

Related U.S. Application Data
(60) Provisional application No. 61/257,054, filed on Nov. 2, 2009.

(51) Int. Cl. A61K 38/02; A61K 38/47; C07C 211/58; B32B 1/02; C07D 209/18; C07C 271/10; C07J 9/00; A61K 38/23; A61K 47/20

(52) U.S. Cl. 424/94.61; 514/1.1; 514/11.9; 564/428; 514/788; 548/495; 560/33; 552/544; 428/34.1

(57) ABSTRACT

The present invention is directed to stable protein formulations, related methods and uses thereof. In particular, the invention relates to a method of stabilizing therapeutic proteins in aqueous solution.

### FIELD OF THE INVENTION

The present invention is directed to pharmaceutical formulations of therapeutic peptides and proteins, in particular peptides and proteins having a propensity to form aggregates.

### BACKGROUND OF THE INVENTION

The development of a large variety of therapeutic proteins and peptides, notably through the progresses in gene recombinant technologies, has to face severe safety and efficacy problems implying the delicate understanding and control of protein misfolding. In particular, protein aggregation has been for a long time a recurrent problem to address when developing biopharmaceuticals (Cleland et al., 1993, Crit. Rev. Ther. Drug. Carrier Syst., 10, 307-377). The formation of protein and peptide aggregates ends up in a broad panel of drawbacks for the producer and the patients spanning from affecting the elegance of the product, its shelf stability, increasing the severity of potential side effects to the rendering of the substance completely unsuitable for use.

Therapeutic protein and peptide aggregation is also a source of batch to batch variabilities in the production chain and its control leads to regulatory and quality control burden which have extremely costly consequences.

Further, aggregation propensity of biopharmaceuticals affects their stability in storage, including shelf-life and their useable administration time, once removed from optimum storage conditions which often undesirably impose restrictions on their conditioning and administration protocol.

Among potential side effects often associated with the use of biopharmaceuticals having a propensity to aggregate, the decrease in pharmacokinetics of the protein or peptide, the enhancement of the immune response to the protein or peptide and toxicity of aggregates are widely known (Rosenberg, 2006, The AAPS Journal, 8(3), E501-E507; Demeule et al., 2006, Eur. J. Pharm. Biopharm., 62:121-30; Bucciantini et al., 2004, J. Biol. Chem., 279:31374-31382). The formation of protein or peptide aggregate-induced antibodies often inhibits drug efficacy and may cause life-threatening complications, especially when directed against endogenous proteins.

PEGylation technology is one of the strategies used in the pharmaceutical industry to improve the pharmacokinetic, pharmacodynamic, and immunological profiles of biopharmaceuticals, and thus enhance their therapeutic effects. This technology involves the covalent attachment of polyethylene glycol (PEG) to a drug and thereby changes the physical and chemical properties of the host biomedical molecule, electrostatic binding, and hydrophobicity, and results in an improvement in the pharmacokinetic profile of the drug.

Currently, PEGylation is used to modify proteins, peptides, oligonucleotides, antibody fragments, and small organic molecules. In general, PEGylation improves drug solubility and decreases immunogenicity, increases drug stability and the retention time of the conjugates in blood, and reduces proteolysis and renal excretion, thereby allowing a reduced dosing frequency (Veronese et al., 2008, Biodrugs, 22(5), 315-29; Bailon et al., 2009, Expert Opin. Drug Deliv., 6(1), 1-16). However, the use of PEGylation technology faces some limitations or drawbacks such as being dependent on the presence of specific amino acids in the sequence of the target protein or peptide, implying covalent modifications of the primary structure of the protein, which may also affect its secondary structure and/or its biological activity, involving the use of reactants such as thiols which remain present in the medium as reactive residues after the protein coupling steps and may crosslink with the protein.

Since stability is a major issue for the production, formulation and/or administration of therapeutic proteins and peptides, as protein and peptide instability such as aggregate formation can lead to loss of biological activity, loss of solubility and even increased immunogenicity, the development of a method of stabilizing and/or stable formulations of proteins and peptides, for example for proteins and peptides having a propensity to aggregate that would lead to an increased stability of those bioproducts would be highly desirable.

### SUMMARY OF THE INVENTION

The invention relates to the unexpected finding of the non-covalent stabilization of proteins such as instable proteins, in particular those having a high propensity to aggregate when formulated in liquid solution, notably in the form of a formulation suitable for administration to a mammal. The invention further relates to the unexpected finding of the stabilizing effects of PEG derivatives on proteins and peptides such as therapeutic proteins and peptides when used in a non-covalent combination, e.g., down to PEG excipients/protein ratios below unity in a process for the preparation of such proteins. Stabilizing effects of proteins according to the invention are supported in particular by the observed reduced propensity of those proteins to form aggregates.

A first aspect of the invention provides a stable protein formulation, said formulation comprising a non-covalent combination of an aqueous carrier, a protein and a PEG derivative, wherein the PEG derivative comprises at least one polyethylene glycol moiety covalently grafted to a hydrophobic group.

A second aspect of the invention provides a pharmaceutical formulation such as a formulation formulated for administration to a mammal (e.g. human) comprising a stable protein formulation according to the invention or a stabilized protein according to the invention.

A third aspect of the invention provides a pharmaceutical unit dosage form suitable to a mammal comprising formulation according to the invention.

A fourth aspect of the invention provides a kit comprising in one or more container(s) a formulation according to the invention together with instruction of use of said formulation.

A fifth aspect of the invention provides a formulation according to the invention for use as a medicament.

A sixth aspect of the invention provides a formulation according to the invention for the prevention or treatment of a disease or a disorder.

A seventh aspect of the invention provides a method of stabilizing a protein or peptide in aqueous solution.

An eighth aspect of the invention provides a process for the preparation of a protein or peptide in aqueous solution or a formulation thereof according to the invention.

A ninth aspect of the invention provides a stabilized protein or peptide or a formulation thereof obtainable by a process or a method according to the invention.

A tenth aspect of the invention provides a method of preventing, treating or ameliorating a disease or a disorder, said method comprising administering in a subject in need thereof a prophylactic or therapeutically effective amount of a formulation according to the invention or of a stabilized protein or peptide according to the invention.

An eleventh aspect of the invention provides a use of a formulation according to the invention or of a stabilized protein or peptide according to the invention for the preparation of a pharmaceutical formulation for the prevention and/or treatment of a disease or disorder.

A twelfth aspect of the invention provides a process for the preparation of a PEG derivative according to the invention.

A thirteenth aspect provides a PEG derivative according to the invention.

### DESCRIPTION OF THE FIGURES

FIG. 1 shows the stabilizing effect of PEG derivatives according to the invention such as described in Example 2 via aggregation kinetics. A: salmon calcitonin (sCT) alone (x), sCT with dansylamide (⋄) 1:1 molar ratio, sCT with dansyl-mPEG 2 kD (Δ) 1:1 molar ratio, sCT with mPEG-amine 2 kD (□) 1:1 molar ratio measured by fluorescence of nile red at 620 nm in 10 mM sodium citrate buffer pH 6; B: salmon calcitonin (sCT) alone (-), sCT with dansyl-mPEG 2 kD ( - - - ) 1:1 molar ratio, measured by turbidity at 450 nm in 10 mM sodium citrate buffer pH 6.

FIG. 2 shows the stabilizing effect of PEG derivatives according to the invention such as described in Example 2 via aggregation kinetics in the early phase of the experiment. A: salmon calcitonin (sCT) alone (x), sCT with dansylamide (⋄) 1:1 molar ratio, sCT with bis-dansyl-PEG 3 kD (Δ) 1:1 molar ratio, sCT with PEG-diamine 3 kD (□) 1:1 molar ratio measured by fluorescence of nile red at 620 nm in 10 mM sodium citrate buffer pH 6; B: salmon calcitonin (sCT) alone (-), sCT with bis-dansyl-PEG 3 kD ( - - - ) 1:1 molar ratio, measured by turbidity at 450 nm in 10 mM sodium citrate buffer pH 6.

FIG. 3 shows the stabilizing effect of PEG derivatives according to the invention such as described in Example 2 via aggregation kinetics in the early phase of the experiment. A: salmon calcitonin (sCT) alone (x), sCT with Tryptophan-mPEG 2 kDa (Δ) 1:1 molar ratio, sCT with Tryptophan-mPEG 2 kDa (▴) 1:5 molar ratio, sCT with Tryptophan-mPEG 2 kDa (-) 1:10 molar ratio, measured by fluorescence of Nile Red at 620 nm in 10 mM sodium citrate buffer pH 6; B: salmon calcitonin (sCT) alone (x), sCT with Tryptophan-mPEG 2 kDa (Δ) 1:1 molar ratio, sCT with Tryptophan-mPEG 2 kDa (▴) 1:5 molar ratio, sCT with T tophan-mPEG 2 kDa (-) 1:10 molar ratio, measured by turbidity at 500 nm in 10 mM sodium citrate buffer pH 6.

FIG. 4 shows the stabilizing effect of PEG derivatives according to the invention such as described in Example 2 via aggregation kinetics in the early phase of the experiment. salmon calcitonin (sCT) alone ( - - - ) measured by fluorescence of Nile Red at 620 nm, sCT with Tryptophan-mPEG 5 kDa (—) 1:5 molar ratio measured by fluorescence of Nile Red at 620 nm; turbidity at 500 nm of salmon calcitonin (sCT) alone (▴), turbidity at 500 nm of sCT with Tryptophan-mPEG 5 kDa (♦) 1:5 molar ratio. All experiments were done in 10 mM sodium citrate buffer pH 6.

FIG. 5 shows the stabilizing effect of PEG derivatives according to the invention such as described in Example 3 via aggregation kinetics in the early phase of the experiment. Hen egg white lysozyme (HEWL) alone (x), HEWL with phenylbutylamine-mPEG 2 kDa (▴) 1:1 molar ratio, HEWL with phenylbutylamine-mPEG 2 kDa (□) 1:10 molar ratio, measured by turbidity at 500 nm in 50 mM sodium phosphate buffer pH 12.2.

FIG. 6 shows the stabilizing effect of PEG derivatives according to the invention such as described in Example 4 via aggregation kinetics in the early phase of the experiment. Hen egg white lysozyme (HEWL) alone (x), HEWL with cholesterol-PEG 2 kDa (▪) 1:1 molar ratio, HEWL with cholesterol-PEG 5 kDa (Δ) 1:1 molar ratio, measured by turbidity at 500 nm in 50 mM sodium phosphate buffer pH 12.2.

### DESCRIPTION OF THE TABLES

Table 1 shows a list of some PEG compounds.

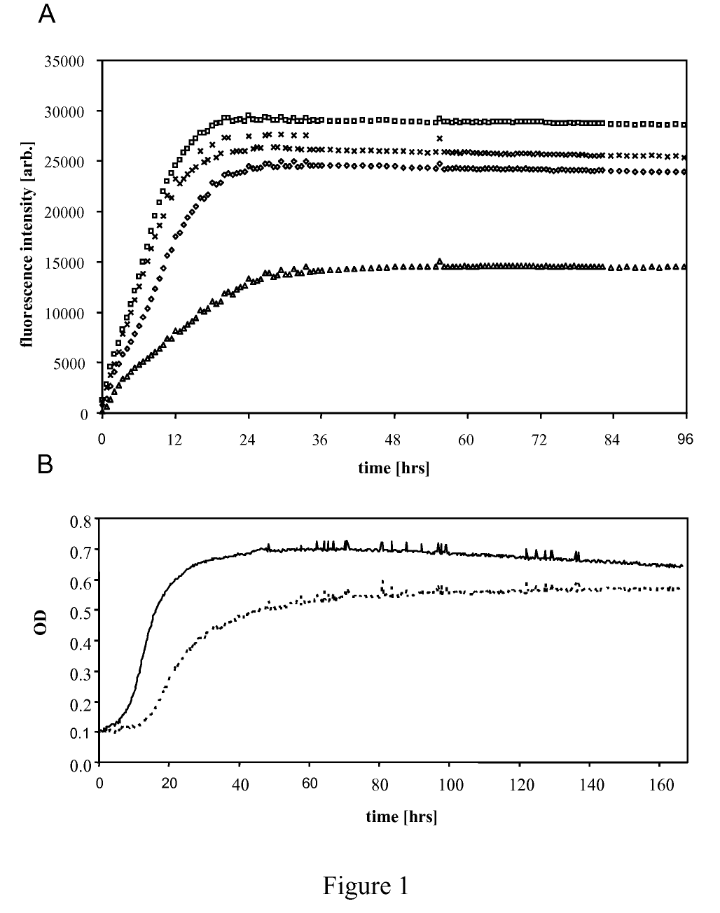

Table 2 shows the optical density (OD) at 450 nm and nile red fluorescence at 620 nm at selected time points during the aggregation kinetics of salmon calcitonin (sCT) alone and sCT with dansyl-mPEG 2 kD in 1:1 molar ratio in 10 mM sodium citrate buffer pH 6 as shown in FIGS. 1A and B.

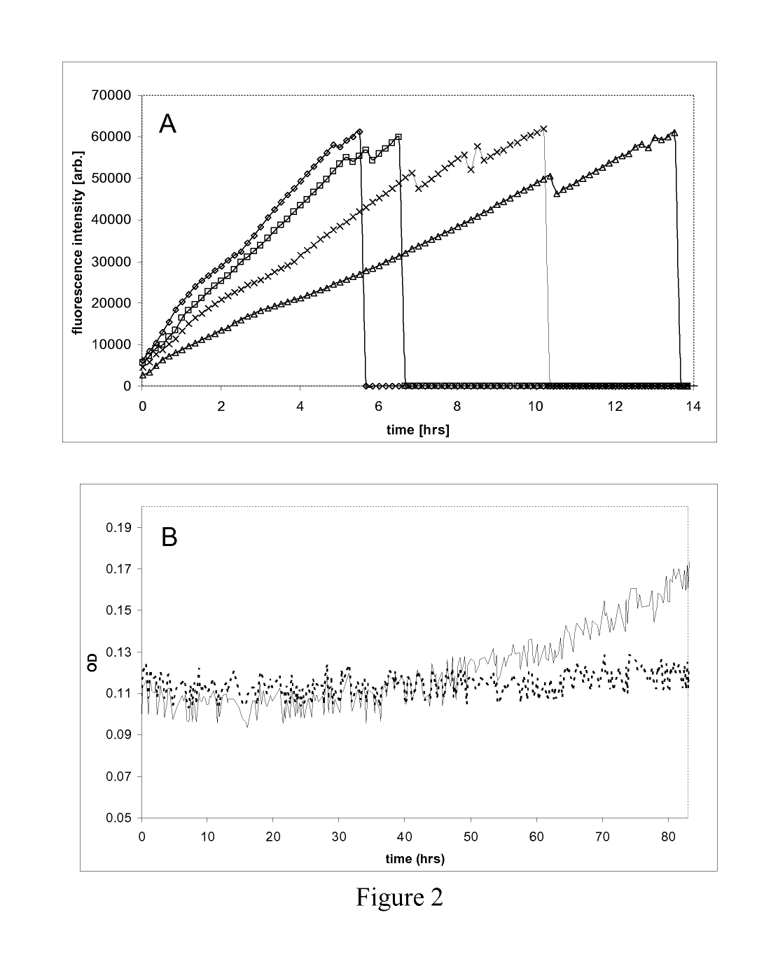

Table 3 shows the optical density (OD) at 450 nm and nile red fluorescence at 620 nm at selected time points during the aggregation kinetics of salmon calcitonin (sCT) alone and sCT with bis-dansyl-PEG 3 kD in 1:1 molar ratio in 10 mM sodium citrate buffer pH 6 as shown in FIGS. 2A and B. A higher sensitivity of the fluorescence detector has been used during measurements of these data compared to those of Table 2 and FIG. 1A.

### DETAILED DESCRIPTION OF THE INVENTION

The term “PEG” or “polyethylene glycol” refers to a polyethylene glycol polymer comprising polymers of the Formula (I): R¹—(OCH₂CH₂)n-X, wherein R¹ is selected from H, optionally substituted C₁-C₆ alkyl such as optionally substituted methyl, optionally substituted ethyl and optionally substituted propyl, such as optionally substituted amino C₁-C₆ alkyl (e.g. 5-Dimethylamino-naphthalene-1-sulfonyl ethylamine); n is selected from 10-500; X is selected from —OR² and —C(O)—OR²; R² is selected from H, optionally substituted heteroaryl, optionally substituted sulfonyl, optionally substituted acyl C₁-C₆ alkyl, optionally substituted alkoxycarbonyl such as para-nitrophenoxycarbonyl and optionally substituted alkoxycarbonyl C₁-C₆ alkyl. In a particular embodiment, “PEG” refers to compounds listed in Table 1 below. Other examples of PEGs are described in Roberts et al., 2002, Adv. Drug Del. Rev. 54, 459-476. In particular, the term includes linear PEGs, such as PEGs of Formula (I), wherein R¹ and R² are H, monofunctional methyl ether PEG (methoxypoly(ethylene glycol)), is abbreviated mPEG (wherein R¹ is CH₃—, X is —OH), branched PEGs having 2 to 10 PEG chains emanating from a central core group such as an amino acid (e.g., lysine), including linear, forked or branched PEGs. Typically, the molecular weight of the PEGs is about 2 to about 50′000 Daltons (e.g. n is selected from 40 to 1200). In a particular embodiment, the molecular weight of the PEGs that can be used in the context of the invention is about 200 to about 20,000 Daltons. In another particular embodiment, the molecular weight of the PEGs is about 500 to about 1′000 Daltons. In yet another embodiment, the molecular weight of the PEGs is about 1′000 to 8′000 Daltons.

TABLE 1

| PEG | Structure | Resulting linkage |
|---|---|---|
| dichlorotriazine-PEG | 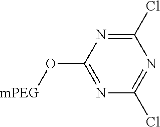 | secondary amine |
| chlorotriazine-PEG | 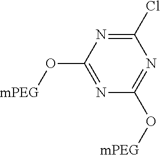 | secondary amine |
| PEG-tresylate | 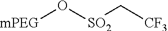 | secondary amine |
| mPEG-acetaldehyde | 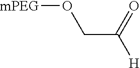 | secondary amine |
| mPEG-succinimidyl carbonate | 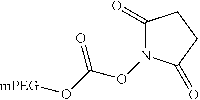 | carbamate/urethane |
| mPEG-benzotriazolyl carbonate | 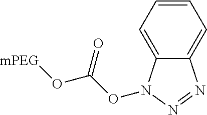 | carbamate/urethane |
| mPEG-p-nitrophenyl carbonate | 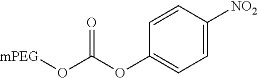 | carbamate/urethane |
| mPEG-2,3,5- trichlorophenyl carbonate | 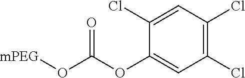 | carbamate/urethane |
| mPEG- carbonylimidazole | 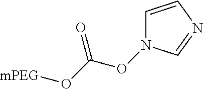 | carbamate/urethane |
| mPEG-succinimidyl succinate | 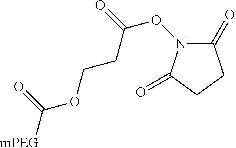 | amide |
| mPEG-aldehyde hydrate | 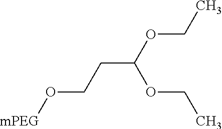 | secondary amine |
| carboxymethylated mPEG | 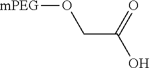 | amide |
| NHS ester of propionic acid mPEG | 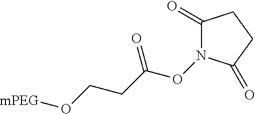 | carbamate |
| NHS ester of α- branched propionic acid mPEG | 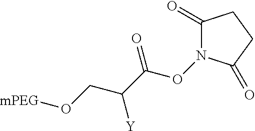 | carbamate |
| thiazolidine-2-thione activated mPEG | 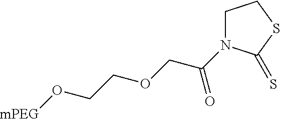 | carbamate |

wherein “Y” represents any branching group.

The term “PEG derivative” refers to a compound comprising at least one polyethylene glycol covalently grafted to a hydrophobic group, wherein the PEG derivative exhibits a stabilizing effect on a protein when combined non-covalently with such protein.

The term “pharmaceutically acceptable derivative” of a specific PEG derivative refers to a PEG derivative which is substituted with from 1 to 5 substituents selected from the group consisting of “C₁-C₆ alkyl”, amino, halogen, cyano, hydroxy, mercapto, nitro, and the like.

The term “pharmaceutically acceptable salts” refers to salts or complexes of the PEG derivatives according to the invention. Examples of such salts include, but are not restricted, to sodium, potassium, ammonium, hydrochloride, magnesium, calcium.

The term “hydrophobic group” comprises any chemical group, which is hydrophobic under following conditions: pH 4-7.5, temperatures between 4° C. and 100° C., water, buffer systems used for protein formulations, ethanol and other organic solvents, for example such that its hydrophobicity, expressed as log D is of about 0 to about 8 (Testa et al. , 2001, Pharmacokinetic Optimization in Drug Research. Biological, physicochemical, and computational strategies. Editor: Pekka Jäckli, Verlag Helvetica Chimica Acta, Zürich, Switzerland and Wiley-VCH, Weinheim, Germany). Examples of hydrophobic groups include naphthylamine sulphonic acid groups such as dansylamide, benzyl groups such as benzyl amine, benzyl alcohol, benzyl amide, phenylbutylamine, phenylbutylamide, steroid groups such as cholesterol, triterpenes, saponins, steroid hormones, amino acids such as tryptophan, phenylalanine, leucine, isoleucine, tyrosine, proline, methionine, alanine and peptides thereof. Examples of peptides as hydrophobic groups according to the invention typically range from about 2 to about 50 amino acids. The grafting of a hydrophobic group to a polyethylene glycol to lead to a PEG derivative according to the invention can be obtained through the reaction of a PEG according to the invention (e.g. a polyethylene glycol to wherein the OH side has been activated) with a hydrophobic group as described below.

In a particular embodiment, a PEG derivative refers to at least one polyethylene glycol covalently grafted to a hydrophobic group selected from dansylamide, phenylbutylamine, cholesterol and an amino acid such as tryptophan.

In another particular embodiment, a PEG derivative refers to at least one polyethylene glycol covalently grafted to a hydrophobic group selected from phenylbutylamine, cholesterol and an amino acid such as tryptophan. In a further particular embodiment, a PEG derivative refers to compounds of Formula (II): R¹—(OCH₂CH₂)—R³, wherein R³ is selected from OR⁴ wherein R⁴ is selected from substituted heteroaryl such as optionally substituted indolyl or optionally substituted napthyl or optionally substituted cyclopentanaphthalenyl groups (e.g. 3-(1,5-Dimethyl-hexyl)-3a,6,6-trimethyl-2,3,3a,4,5,5a,6,9,9a,9b-decahydro-1H-cyclopenta[a]naphthalene), substituted amide (e.g. formylamino-(1H-indo1-3-yl)-acetic acid or N-(4-Phenyl-butyl)-formamide), and substituted amine such as optionally substituted sulfonyl amino (e.g. 5-dimethylamino-naphthalene-1-sulfonyl amine); n is selected from 40 to 122; R¹ is as defined above. In a particular embodiment, R¹ is methyl.

In another particular embodiment, R¹ is H.

In another particular embodiment, “PEG derivative” refers to compounds selected from the group consisting of:

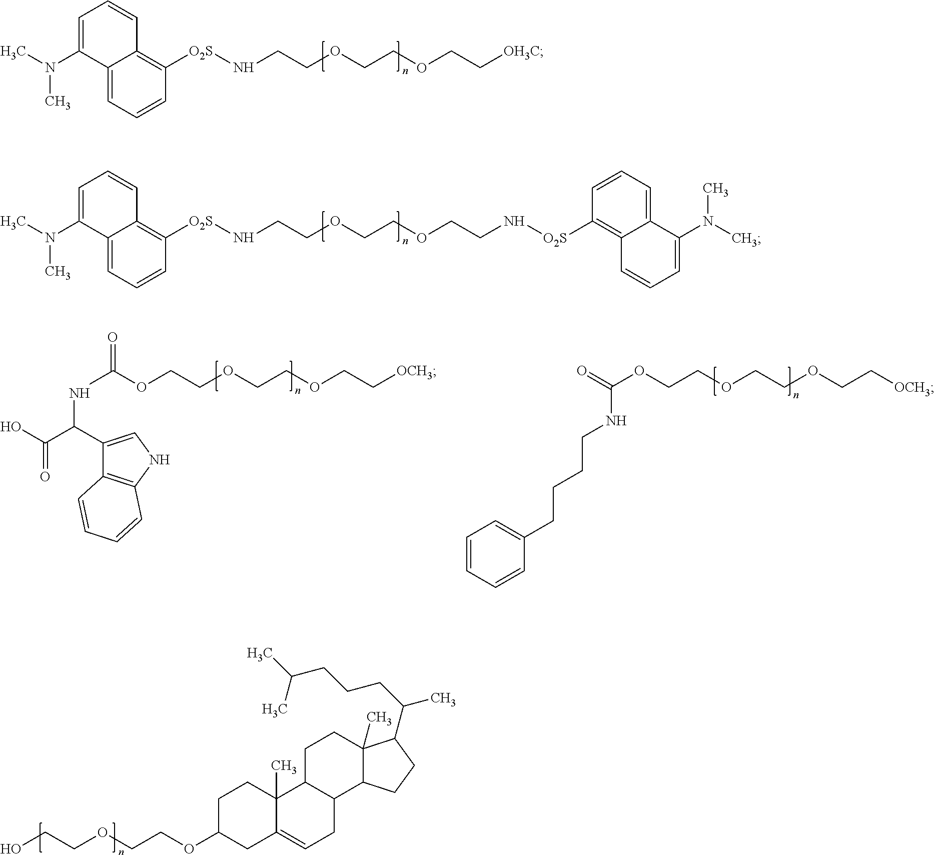

and any pharmaceutically acceptable salts, pharmaceutically acceptable derivatives or isomers thereof.

In another particular embodiment, “PEG derivative” refers to compounds selected from the group consisting of:

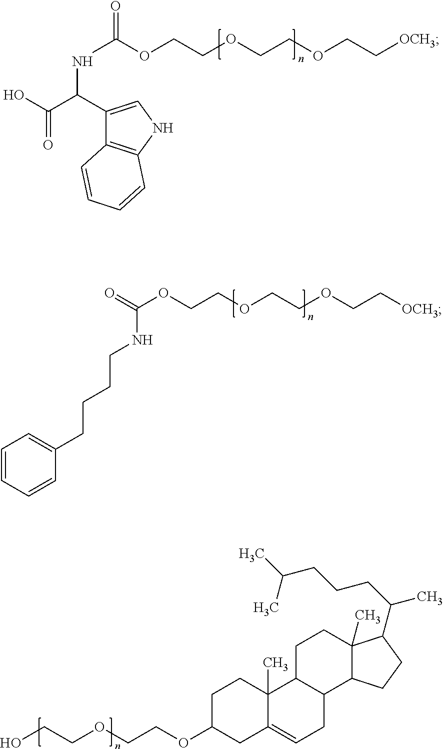

and any pharmaceutically acceptable salts, pharmaceutically acceptable derivatives or isomers thereof. Synthesis of PEG derivatives of the invention may be carried out by known methods, for example as described in U.S. Pat. No. 5,286,637 or Miyajima et al., 1987, Colloid Polym. Sci., 265, 943.

The term “stabilized protein” refers to a protein stabilized by a method according to the invention.

The term “C₁-C₆ alkyl” when used alone or in combination with other terms, comprises a straight chain or branched C₁-C₆ alkyl which refers to monovalent alkyl groups having 1 to 6 carbon atoms.

The term “alkoxy C₁-C₆ alkyl” refers to C₁-C₆ alkyl groups having an alkoxy substituent, including methoxyethyl and the like.

The term “heteroaryl” refers to a monocyclic heteroaromatic, or a bicyclic or a tricyclic fused-ring heteroaromatic group. For example, heteroaryl refers to indolyl, or napthyl or cyclopentanaphthalenyl groups.

The term “acyl C₁-C₆ alkyl” to C₁-C₆ alkyl groups having an acyl substituent, including 2-acetylethyl and the like.

The term “sulfonyl” refers to group “—SO₂—R” wherein R is selected from “aryl,” “heteroaryl,” “C₁-C₆ alkyl,” “C₁-C₆ alkyl” substituted with halogens, e.g., an —SO₂—CF₂ group, “C₂-C₆ alkenyl,” “C₂-C₆ alkynyl,” “C₃-C₈-cycloalkyl,” “heterocycloalkyl,” “aryl,” “heteroaryl,” “aryl C₁-C₆ alkyl”, “heteroaryl C₁-C₆ alkyl,” “aryl C₂-C₆ alkenyl,” “heteroaryl C₂-C₆ alkenyl,” “aryl C₂-C₆ alkynyl,” “heteroaryl C₂-C₆ alkynyl,” “cycloalkyl C₁-C₆ alkyl,” or “heterocycloalkyl C₁-C₆ alkyl”.

The term “sulfonylamino” refers to a group —NRSO₂—R′ where R and R′ are independently H, “C₁-C₆ alkyl,” “C₂-C₆ alkenyl,” “C₂-C₆ alkynyl,” “C₃-C₈-cycloalkyl,” “heterocycloalkyl,” “aryl,” “heteroaryl,” “aryl C₁-C₆ alkyl”, “heteroaryl C₁-C₆ alkyl,” “aryl C₂-C₆ alkenyl,” “heteroaryl C₂-C₆ alkenyl,” “aryl C₂-C₆ alkynyl,” “heteroaryl C₂-C₆ alkynyl,” “C₃-C₈-cycloalkyl C₁-C₆ alkyl,” or “heterocycloalkyl C₁-C₆ alkyl”.

The term “alkoxycarbonyl” refers to the group —C(O)OR where R includes “C₁-C₆ alkyl”, “aryl”, “heteroaryl”, “aryl C₁-C₆ alkyl”, “heteroaryl C₁-C₆ alkyl” or “heteroalkyl”.

Unless otherwise constrained by the definition of the individual substituent, the term “substituted” refers to groups substituted with from 1 to 5 substituents selected from the group consisting of “C₁-C₆ alkyl,” “C₂-C₆ alkenyl,” “C₂-C₆ alkynyl,” “C₃-C₈-cycloalkyl,” “heterocycloalkyl,” “C₁-C₆ alkyl aryl,” “C₁-C₆ alkyl heteroaryl,” “C₁-C₆ alkyl cycloalkyl,” “C₁-C₆ alkyl heterocycloalkyl,” “amino,” “aminosulfonyl,” “ammonium,” “acyl amino,” “amino carbonyl,” “aryl,” “heteroaryl,” “sulfinyl,” “sulfonyl,” “alkoxy,” “alkoxy carbonyl,” “carbamate,” “sulfanyl,” “halogen,” trihalomethyl, cyano, hydroxy, mercapto, nitro, and the like.

The term “amphipathic peptide” comprises peptides containing both hydrophilic and hydrophobic amino acid residues, where spatial separation of these residues, such as for example through the secondary structure of the peptide, result in their ability to partition at an interface between a polar and an apolar medium such as a lipidic interface, an air/water interface, hydrophilic solvent/hydrophobic solvent interface and air/packaging material interface. Typically, amphipathic peptides present an amphipathicity defined by a mean hydrophobic moment between about 0 and about 0.9, according to the Eisenberg plot (Eisenberg et al., 1984, J. Mol. Biol. 179, 125-142). Typical amphipathic peptides used in the context of the invention include samples from reference McLean. et al., 1991, Biochemistry 30, 31-37.

The term “protein” includes any natural, synthetic or recombinant protein or peptide, in particular proteins, notably therapeutic proteins (e.g., polypeptides, enzymes, antibodies, hormones) which are unstable in solution such as for example hydrophobic proteins. Typically, molecular weight of the peptides and proteins according to the invention range from about 200 D to about 1′000 kD. Examples of proteins in the context of the invention are salmon calcitonin (sCT), interferon-beta and granulocyte-colony stimulating factor (G-CSF). In another embodiment, an example of a protein according to the invention comprises hen egg white lysozyme (HEWL).

As used herein, “treatment” and “treating” and the like generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it such as a preventive early asymptomatic intervention; (b) inhibiting the disease, i.e., arresting its development; or relieving the disease, i.e., causing regression of the disease and/or its symptoms or conditions such as improvement or remediation of damage.

The term “subject” as used herein refers to mammals. For examples, mammals contemplated by the present invention include human, primates, domesticated animals such as cattle, sheep, pigs, horses, laboratory rodents and the like.

The term “effective amount” as used herein refers to an amount of at least one protein or a pharmaceutical formulation thereof according to the invention that elicits the biological or medicinal response in a tissue, system, animal or human that is being sought. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented. The term also includes herein the amount of active polypeptide sufficient to reduce the progression of the disease thereby elicit the response being sought (i.e. an “inhibition effective amount”).

The term “efficacy” of a treatment according to the invention can be measured based on changes in the course of disease in response to a use or a method according to the invention.

The term “stable” or “stabilized” refers in the context of the invention to formulations in which the protein therein retains its physical stability (e.g. level of aggregation or aggregation propensity decreased, absence of precipitation or denaturation) and/or chemical stability (e.g. absence of chemically altered forms by disulfide bond formation or exchange) upon formulation or storage. Stability of the protein formulations according to the invention may be measured by various techniques known to the skilled person in the art. For example, stability can be measured by aggregation state measurements (e.g., by field flow fractionation, light scattering, high performance size exclusion, ultracentrifugation, turbidity measurements, fluorescence microscopy, electron microscopy, others named in Mahler et al., 2008, J. Pharm. Sci., 98(9):2909-2934. Preferably, the stability of the formulation is measured at a selected temperature and/or for a selected period of time storage.

The term “stabilizing amount” according to the invention refers to an amount of at least one PEG derivative according to the invention that elicits the stabilizing effect on a protein. The stabilizing effect of a PEG derivative or a method according to the invention on a protein can be measured by a reduction in the rate and extent of aggregation of the protein once non-covalently combined with a PEG derivative according to the invention, such as described in (Capelle et al., 2009, Pharm. Res., 26 :118-128). Alternatively, the stabilizing effect of a PEG derivative or a method according to the invention on a protein can be measured by an increased bioavailability and/or a decrease of immunogenicity of the protein once non-covalently combined with a PEG derivative according to the invention, such as described in Graham, 2003, Adv. Drug Del. Rev., 55: 1293-1302 or Caliceti et al. 2003, Adv. Drug Del. Rev., 55: 1261-1277.

The term “pharmaceutical formulation” refers to preparations which are in such a form as to permit biological activity of the active ingredient(s) to be unequivocally effective and which contain no additional component which would be toxic to subjects to which said formulation would be administered.

### PEG Derivatives According to the Invention

According to an embodiment, is provided a PEG derivative according to the invention wherein said at least one polyethylene glycol is covalently grafted to a hydrophobic group, wherein the PEG is above defined. In a particular embodiment, PEG is selected from m-PEGs, in particular m-PEGs of molecular weight of 2 kDa or 3 kDa. In another particular embodiment, PEG is an m-PEG of molecular weight of 5 kDa.

According to another embodiment, is provided a PEG derivative according to the invention wherein the hydrophobic group is selected from groups having a log D between 0 and 8. In another particular embodiment, the hydrophobic group is a dansyl group (DNS). In another particular embodiment, the hydrophobic group is selected from phenylbutylamine, cholesterol and an amino acid such as tryptophan.

### Formulations According to the Invention

According to an embodiment, is provided a stable protein formulation, said formulation comprising a non-covalent combination of an aqueous carrier, a protein and a PEG derivative, wherein the PEG derivative comprises at least one polyethylene glycol moiety covalently grafted to a hydrophobic group.

According to another embodiment, is provided a stabilized protein or a formulation thereof obtainable by a process or a method according to the invention.

According to further embodiment, the invention provides a formulation according to the invention wherein the protein formulation thereof is at a concentration in the range from about 0.01 ng/ml to about 500 mg/ml.

According to another further embodiment, the invention provides a formulation according to the invention wherein the PEG derivative is at a concentration in the range from about 0.001 ng/ml to about 1 g/ml.

According to another further embodiment, the invention provides a formulation according to the invention wherein the molar ratio PEG derivative to protein is in the range from about 1:0.001 molar ratio to about 1:1′000.

According to another further embodiment, the invention provides a formulation according to the invention wherein the molar ratio PEG derivative to protein is in the range from about 1:1 molar ratio to about 1:100.

According to another further embodiment, the invention provides a formulation according to the invention wherein the molar ratio PEG derivative to protein is 1:1.

According to another further embodiment, is provided a stable protein formulation according to the invention wherein the PEG derivative is an mPEG.

According to another further embodiment, is provided a stable protein formulation according to the invention wherein the PEG derivative is an mPEG of molecular weight of 2 kDa.

According to another further embodiment, is provided a stable protein formulation according to the invention wherein the PEG derivative is an mPEG of molecular weight of 5 kDa.

According to another further embodiment, is provided a stable protein formulation according to the invention wherein the PEG derivative is such that the said at least one polyethylene glycol moiety is covalently grafted to a hydrophobic group selected from dansylamide, tryptophan, phenylbutylamine, cholesterol, and an amphipathic peptide.

According to another further embodiment, is provided a stable protein formulation according to the invention wherein the PEG derivative is such that the said at least one polyethylene glycol moiety is covalently grafted to a hydrophobic group selected from tryptophan, phenylbutylamine and cholesterol.

According to another further embodiment, is provided a stable protein formulation according to the invention wherein the PEG derivative is of Formula (II): R¹—(OCH₂CH₂)ₙ—R³, wherein R³ is selected from OR⁴ wherein R⁴ is selected from substituted heteroaryl such as optionally substituted indolyl or optionally substituted napthyl or optionally substituted cyclopentanaphthalenyl groups (e.g. 3-(1,5-Dimethyl-hexyl)-3a,6,6-trimethyl-2,3,3a,4,5,5a,6,9,9a,9b-decahydro-1H-cyclopenta [a]naphthalene), substituted amide (e.g. formylamino-(1H-indo1-3-yl)-acetic acid or N-(4-phenyl-butyl)-formamide), and substituted amine such as optionally substituted sulfonyl amino (e.g. 5-dimethylamino-naphthalene-1-sulfonyl amine); n is selected from 40 to 120; R¹ is as defined above.

In another particular embodiment, is provided a stable protein formulation according to the invention wherein the PEG derivative is selected from the group consisting of:

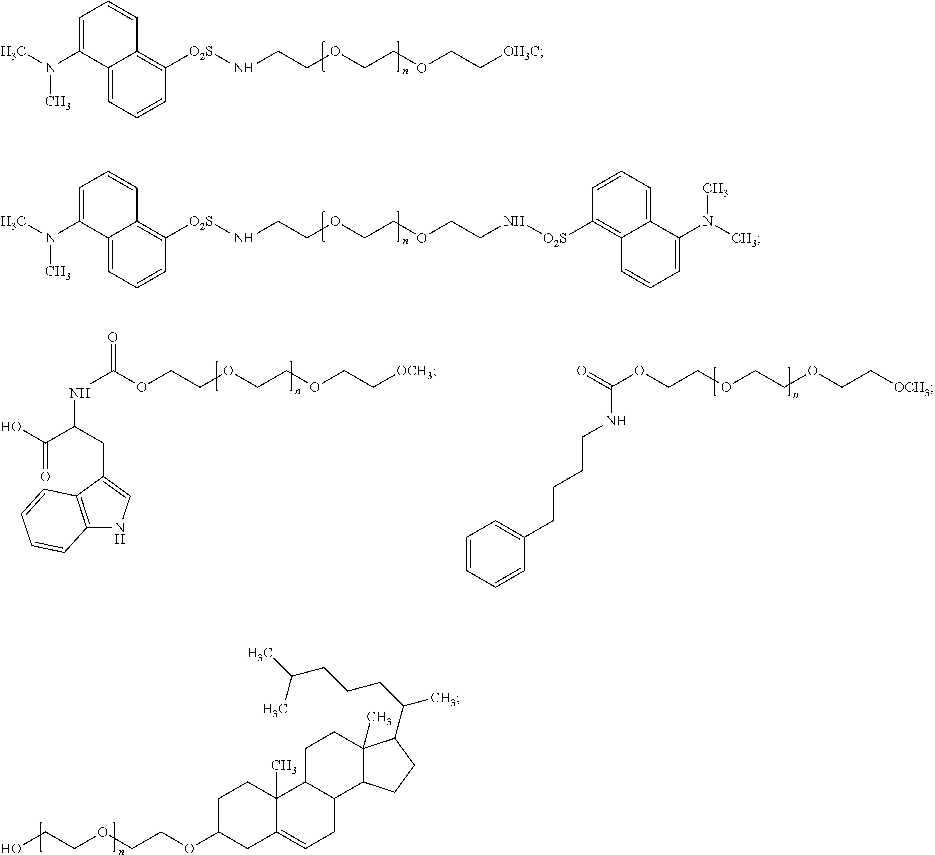

and any pharmaceutically acceptable salts, pharmaceutically acceptable derivatives or isomers thereof.

In another particular embodiment, is provided a stable protein formulation according to the invention wherein the PEG derivative is selected from the group consisting of:

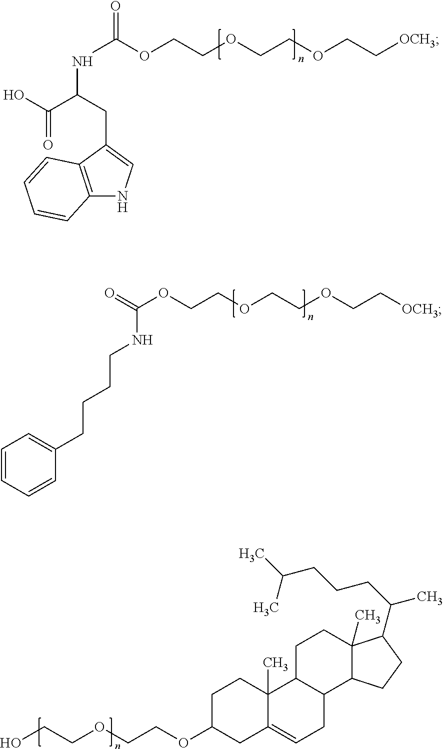

and any pharmaceutically acceptable salts, pharmaceutically acceptable derivatives or isomers thereof.

According to another further embodiment, is provided a stable protein formulation according to the invention wherein the protein is selected from sCT and HEWL and the PEG derivative is selected from the group consisting of:

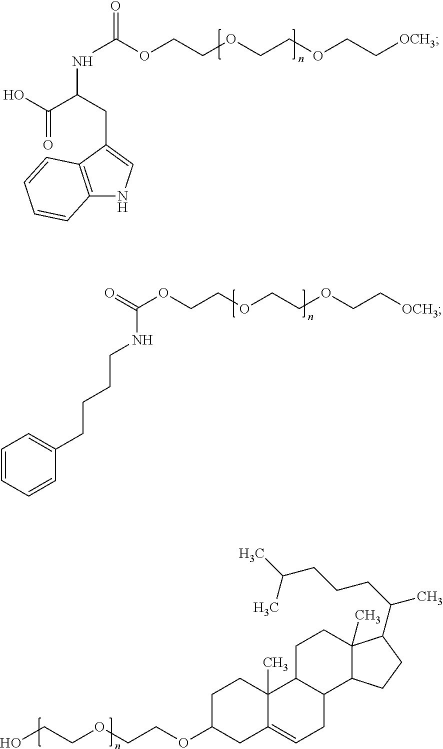

and any pharmaceutically acceptable salts, pharmaceutically acceptable derivatives or isomers thereof

According to another further embodiment, the invention provides a formulation according to the invention further comprising an excipient.

According to a further embodiment, the invention provides a formulation according to the invention wherein the formulation is a pharmaceutical formulation, notably formulated for administration in a mammal, typically a human mammal.

According to another further embodiment, the invention provides a kit comprising in one or more container a formulation according to the invention together with instruction of use of said formulation.

According to another further embodiment, the invention provides a kit for reconstituting a protein in solution comprising in one container a lyophilized protein, notably a therapeutic protein, and a PEG derivative of the invention in another container or another part of said container, optionally together with a container containing a sterile buffer for reconstituting the protein and optionally with instruction of use of said kit.

According to another further embodiment, the invention provides a formulation according for use as a medicament.

In another particular embodiment, is provided a PEG derivative according to the invention, wherein the PEG derivative is:

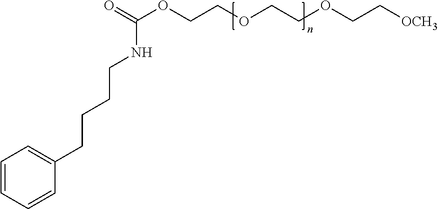

and any pharmaceutically acceptable salts, pharmaceutically acceptable derivatives or isomers thereof.

Compositions or formulations according to the invention may be administered as a pharmaceutical formulation, which can contain one or more protein according to the invention in any form described herein. Formulations of this invention may further comprise one or more pharmaceutically acceptable additional ingredient(s) such as alum, stabilizers, antimicrobial agents, buffers, coloring agents, flavoring agents, adjuvants, and the like.

Formulations of the invention, together with a conventionally employed adjuvant, carrier, diluent or excipient may be placed separately into the form of pharmaceutical compositions and unit dosages thereof, and in such form may be employed as liquids such as solutions, suspensions, emulsions, elixirs, or capsules filled with the same, all in the form of sterile injectable solutions. Such pharmaceutical compositions and unit dosage forms thereof may comprise ingredients in conventional proportions, with or without additional active compounds or principles, and such unit dosage forms may contain any suitable effective amount of the active ingredient commensurate with the intended daily dosage range to be employed.

Such liquid preparations may contain additives including, but not limited to, suspending agents, emulsifying agents, non-aqueous vehicles and preservatives. Suspending agent include, but are not limited to, sorbitol syrup, methyl cellulose, glucose/sugar syrup, gelatin, hydroxyethyl cellulose, carboxymethyl cellulose, aluminum stearate gel, and hydrogenated edible fats. Emulsifying agents include, but are not limited to, lecithin, sorbitan monooleate, and acacia. Injectable compositions are typically based upon injectable sterile saline or phosphate-buffered saline or other injectable carriers known in the art.

In another particular aspect, the formulation is adapted for delivery by repeated administration.

Further materials as well as formulation processing techniques and the like are set out in Part 5 of Remington's Pharmaceutical Sciences, 21^{st} Edition, 2005, University of the Sciences in Philadelphia, Lippincott Williams & Wilkins, which is incorporated herein by reference.

Formulations according to the invention, stabilized protein and formulations thereof obtainable by a process or a method according to the invention are useful in the prevention and/or treatment of a disease or a disorder.

### Methods of Preparation According to the Invention

According to one aspect of the invention, is provided a method of stabilizing a protein in aqueous solution by non-covalently combining said protein with a PEG derivative according to the invention.

According to another embodiment, is provided a process for the preparation of a protein or a formulation thereof comprising the steps of:

(i) non-covalently combining said protein with a PEG derivative into a liquid mixture or forming said protein in a liquid medium containing a PEG derivative, wherein the said PEG derivative comprises at least one polyethylene glycol moiety covalently grafted to a hydrophobic group;

(ii) collecting the liquid mixture or liquid medium obtained under step (i) containing the stabilized protein non-covalently combined with the said PEG derivative, wherein the percentage of monomers of protein is increased as compared to said protein prepared in absence of the said PEG derivative.

Typically, for a PEG derivative being PEG-DNS, the percentage of aggregates of stabilized protein formulation is reduced by about at least 30% after ca. 7 days at 26° C. at 2.5 mg/ml. Typically, for a PEG derivative being PEG-Trp, the percentage of aggregates of stabilized sCT formulation is reduced by about at least 90% after ca. 2.5 days at 26° C. at 2.5 mg/ml for a molar ratio sCT/PEG derivative of 1:10.

Typically, for a PEG derivative being Cholesteryl-PEG, the percentage of aggregates of stabilized protein formulation is reduced by about at least 100%. For a PEG derivative being phenylbutylamine, the onset of the aggregation process is shifted of about at least 3.5 hours for a molar ratio HEWL/PEG derivative of 1:10.

In a particular embodiment, is provided a method according to the invention wherein the said PEG derivative is an mPEG derivative.

According to another further embodiment, is provided a method according to the invention wherein the said PEG derivative is an mPEG derivative of molecular weight of 2 kDa.

According to another further embodiment, is provided a method according to the invention wherein the said PEG derivative is an mPEG derivative of molecular weight of 5 kDa.

In a particular embodiment, is provided a method according to the invention wherein the said PEG derivative is such that the said at least one polyethylene glycol covalently grafted to a hydrophobic group selected from dansylamide, tryptophan, phenylbutylamine, cholesterol, and an amphipathic peptide. In a particular embodiment, the hydrophobic group is selected from phenylbutylamine, dansylamide, cholesterol and an amino acid such as tryptophane.

In a further embodiment, the invention provides a method or a process according to the invention wherein the aqueous solution is a pharmaceutical formulation and the protein is in a therapeutically effective amount.

In a further embodiment, the invention provides a method, a process, a use or a formulation according to the invention wherein the protein is selected from sCT and HEWL.

In a further aspect of the invention, the method or process according to the invention may be useful in decreasing the aggregation ability of a protein during its production process.

In another aspect the method or process according to the invention may be useful in preparing stable formulations of proteins presenting an increased shelf-life and enabling multiple dosing conditioning.

In another aspect is provided a process for the preparation of a PEG derivative according to the invention comprising the step of reacting an mPEG-p-nitrophenyl carbonate with phenylbutylamine in an anhydrous solvent, typically selected from dichloromethane, chloroform, Dimethylformamide (DMF) and Dimethyl Sulfoxide (DMSO) at a pH between about 9 and about 11 at room temperature.

### Mode of Administration

Formulations of this invention may be administered in any manner including parenterally, transdermally, rectally, transmucosally, intra-ocular or combinations thereof. Parenteral administration includes, but is not limited to, intravenous, intra-arterial, intra-peritoneal, subcutaneous, intramuscular, intra-thecal, and intra-articular. The compositions of this invention may also be administered in the form of an implant, which allows slow release of the compositions as well as a slow controlled i.v. infusion.

### Methods According to the Invention

According to another aspect, the invention provides a method of preventing, treating or ameliorating a disease or a disorder, said method comprising administering in a subject in need thereof a prophylactic or therapeutically effective amount of a stable protein formulation or a formulation of a stabilized protein obtainable by a process or a method according to the invention.

The dosage administered, as single or multiple doses, to an individual will vary depending upon a variety of factors, including pharmacokinetic properties, patient conditions and characteristics (sex, age, body weight, health, size), extent of symptoms, concurrent treatments, frequency of treatment and the effect desired.

### Patients

In an embodiment, patients according to the invention are patients suffering from a disease or a disorder for which the protein of the invention is therapeutically beneficial. The stabilized formulation according to the invention of the said protein allows the use of lower doses of said protein, and/or increases the protein therapeutic efficacy and/or leads to a decrease in side effects as compared to the protein administered in the form of known formulations.

References cited herein are hereby incorporated by reference in their entirety. The present invention is not to be limited in scope by the specific embodiments and drawings described herein, which are intended as single illustrations of individual aspects of the invention, and functionally equivalent methods and components are within the scope of the invention. The examples illustrating the invention are not intended to limit the scope of the invention in any way.

### EXAMPLES

### General Procedures & Conditions

The following studies are conducted to support the influence of a PEG derivative according to the invention on the stability of proteins. Aggregation (reduction or absence of which) of the protein is measured to determine whether its non-covalent association with a PEG derivative according to the invention into a single formulation influences the aggregation state of this protein. Since aggregates have been observed to cause severe side-effects, this study is of great importance for anticipating beneficial effects in clinical use. Further, bioavailability and immunogenicity studies are conducted to support further stabilizing effects.

The following abbreviations refer respectively to the definitions below:

a.u. (arbitrary units), hr (hours), i.v. (intravenous), kD or kDa (kilo Dalton), MHz (Megahertz), mM (millimolar), nm (nanometer), ppm (parts per million), qs (quantum satis), s.c. (subcutaneous) Ar (aromatic), FFF (flow field-flow fractionation), DMSO (Dimethyl Sulfoxide), DNS (Dansyl), DANSA (dansylamide), FTIR (Fourier Transform Infrared), LS (light scattering), MS (mass spectrometry), NMR (Nuclear Magnetic resonance), OD (optical density), TFA (Trifluoroacetic acid), UV (Ultraviolet).

### Example 1

### Synthesis of DNS-PEG Derivatives

The following PEG derivatives according to the invention (of Formula (II), wherein R³ is substituted sulfonyl amino (e.g. 5-dimethylamino-naphthalene-1-sulfonyl amine); n is selected from 40 to 120 and R¹ is optionally substituted C₁-C₆ alkyl (e.g. methyl) or substituted amino C₁-C₆ alkyl (e.g. 5-Dimethylamino-naphthalene-1-sulfonyl ethylamine), respectively) were synthesized as follows:

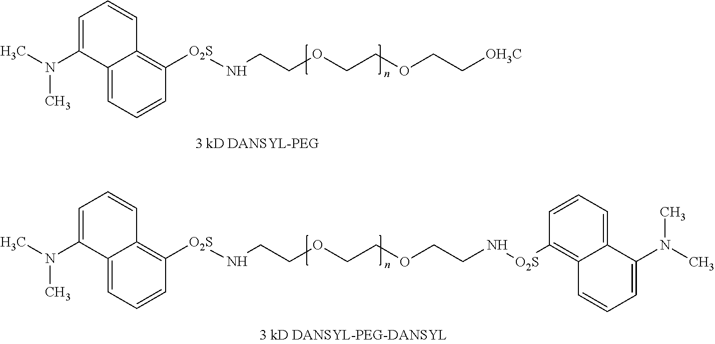

Synthesis of dansyl-PEG 2 kDa (Method Adapted from Pendri et al., 1995, Bioconjugate Chem., 6, 596)

0.33 mMol of dried mPEG-amine 2 kDa (Iris Biotech, Germany) were dissolved in 34 ml of anhydrous toluene and 0.98 mMoles of dansyl chloride and 0.13 mMoles of dry triethyl amine were added. The reaction was performed at 100° C. under reflux for 24 hours. Toluene was evaporated and the solid was redissolved in dichloromethane. After precipitation from cold diethyl ether, the solid was collected via filtration and reprecipitated from isopropyl alcohol. A slightly yellowish powder was obtained that was dried under vacuum and characterized by NMR, UV and FTIR spectrometry. ¹H-NMR (300 MHz, DMSO-d-6): 2.82 ppm, CH₃—N-(s); 2.96 ppm, CH₃—N-(s); 3.23 ppm, CH₃—O-(s); 3.50 ppm, —O—CH₂-(s); 7.27 ppm, aromatic (d); 7.60 ppm, aromatic (t); 8.10 ppm, aromatic (d); 8.36 ppm, aromatic (d); 8.45 ppm, aromatic (d). ¹³C-NMR (300 MHz, DMSO-d-6): 42.04 ppm, CH₃—N-(s); 44.90 ppm, CH₃—N-(s); 57.85 ppm, CH₃—O-(s); 69.59 ppm, —O—CH₂-(m); 114.89 ppm, aromatic (s); 119.08 ppm, aromatic (s); 123.41 ppm, aromatic (s); 127.80 ppm, aromatic (s); 128.99 ppm, aromatic (s); 136.14 ppm, aromatic (s); 151.12 ppm, aromatic (s).

Synthesis of bis-dansyl-PEG 3 kDa (Method Adapted from Pendri. et al., 1995, Above)

0.033 mMol of dried PEG-diamine 3 kDa (Iris Biotech, Germany) were dissolved in 30 ml of anhydrous toluene and 0.2 mMoles of dansyl chloride and 0.27 mMoles of dry triethyl amine were added. The reaction was performed at 100° C. under reflux for 24 hours. Toluene was evaporated and the solid was redissolved in dichloromethane. After precipitation from cold diethyl ether, the solid was collected via filtration and reprecipitated from isopropyl alcohol. A slightly yellowish powder was obtained that was dried under vacuum and characterized by NMR, UV and FTIR spectrometry. ¹H-NMR (300 MHz, DMSO-d-6): 2.83 ppm, CH₃—N-(s); 2.95 ppm, CH₃—N-(s); 3.23 ppm, CH₃—O-(s); 3.50 ppm, —O—CH₂-(s); 3.50 ppm, —O—CH₂—; 7.24 ppm, aromatic (d); 7.59 ppm, aromatic (t); 8.10 ppm, aromatic (d); 8.28 ppm, aromatic (d); 8.45 ppm, aromatic (d). ¹³C-NMR (300 MHz, DMSO-d-6): 42.43 ppm, CH₃—N-(s); 45.27 ppm, CH₃—N-(s); 69.98 ppm, —O—CH₂-(m); 115.27 ppm, aromatic (s); 119.47 ppm, aromatic (s); 123.77 ppm, aromatic (s); 128.01 ppm, aromatic (s); 129.36 ppm, aromatic (s); 136.56 ppm, aromatic (s); 151.49 ppm, aromatic (s).

### Example 2

### Comparison of the Aggregation Propensity of Calcitonin Alone and in Combination with DNS-mPEGs

In order to assess the stabilizing effect of PEG derivatives according to the invention, the aggregation propensity of salmon calcitonin (sCT) is assayed in presence or absence of PEG derivatives according to the invention. Salmon calcitonin is a 32-amino acid polypeptide hormone (Martha et al., 1993, Biotechnology, 11, 64-70). It acts to reduce blood calcium (Ca²⁺), is used for the treatment of various bone associated disorders (Capelle et al., 2009, Pharm. Res., 26: 118-128) and has a lower propensity to aggregate in solution than the human form (Gaudiano et al., 2005, Biochim Biophys Acta 1750:134-145).

Aggregation of Salmon Calcitonin (sCT)

Salmon calcitonin (Therapeomic Inc., Switzerland) in a final concentration of 2.5 mg/ml per well with and without the respective excipients to be tested, i.e. non-conjugated mPEG-amines and non-conjugated hydrophobic headgroups were prepared in 4 different buffer systems: 10 mM sodium acetate pH 5, 10 mM sodium citrate pH 5, 10 mM sodium citrate pH 6, 10 mM sodium phosphate buffer pH 8. Samples are prepared two times and nile red in a final concentration of 1 μM is added to one of each. Aggregation is followed in UV-transparent 96-well plates or 384-well Costar® plates from Corning (Corning Life Sciences, Schiphol, Netherlands) by a microplate reader (Tecan Safire™ microplate reader, Tecan Group Ltd, Männedorf, Switzerland) by monitoring turbidity, nile red fluorescence and intrinsic fluorescence of the protein/peptide drug or the hydrophobic head-group. After finishing the aggregation kinetics, final spectra of nile red fluorescence, UV and protein/peptide drug or hydrophobic head group fluorescence were measured.

Aggregation Propensity of Calcitonin Alone and in Combination with mPEGs

Salmon calcitonin (sCT) was aggregated using different buffer systems in which sCT was shown to be unstable (Capelle et al., 2009, Pharm. Res., 26:118-128). Aggregation was checked in absence of any excipient, in presence of equimolar amounts of dansylamide, mPEG-amine 2 kD and dansyl-mPEG 2 kD, respectively. Lower aggregation was seen for the equimolar mixture of sCT with dansyl-mPEG 2 kD in citrate buffer pH 6 by checking nile red fluorescence at 620 nm over time (FIG. 1A, 2A) and turbidity at 450 nm (FIG. 1B, 2B). It can be clearly seen by both techniques that the final level of aggregation is lower. Furthermore, turbidity shows that the onset of aggregation has been prolonged. The same tendency was observed in phosphate buffer pH 8.

TABLE 2

| | | | | Nile red fluorescence at | |
|---|---|---|---|---|---|
| | OD at 450 nm | | | 620 nm | |
| | | sCT + D- | | | sCT + D- |
| time (hrs) | sCT | PEG2 | time (hrs) | sCT | PEG2 |
| 0 | 0.10 | 0.10 | 0 | 1058 | 181 |
| 9.5 | 0.22 | 0.12 | 9.5 | 18581 | 6375 |
| 20 | 0.63 | 0.37 | 20 | 27276 | 11818 |
| 100 | 0.68 | 0.55 | 100 | 25328 | 14469 |
| 166 | 0.64 | 0.57 | 166 | 24729 | 14144 |
| lag time of | 3.5 | 10.9 hours | slope of | 1843 a.u./hrs | 488 a.u./ |
| aggregation | hours | | aggregation | | hrs |
| | | | curve | | |

TABLE 3

| | | | | Nile red fluorescence | |
|---|---|---|---|---|---|
| | | | | at 620 nm | |
| | OD at 450 nm | | | | sCT + |
| | | sCT + bis- | | | bis-D- |
| time (hrs) | sCT | D-PEG3 | time (hrs) | sCT | PEG3 |
| 0 | 0.11 | 0.11 | 0 | 4560 | 2599 |
| 6.2 | 0.15 | 0.13 | 5 | 38483 | 24967 |
| 10 | 0.22 | 0.15 | 10 | 61019 | 49030 |
| 30.5 | 0.49 | 0.39 | 13.5 | >65000 | 61143 |
| 140 | 0.56 | 0.50 | 20 | >65000 | >65000 |
| lag time of | 4.2 | 8.0 hours | slope of | 5546 a.u./hrs | 4351 a.u./ |
| aggregation | hours | | aggregation | | hrs |
| | | | curve | | |

Bis-D-PEG3 = bis-DNS-PEG 3 kDa;
D-PEG2 = DNS-mPEG 2 kDa

Experiments with sCT (salmon calcitonin) formulations according to the invention at various SCT/Dansyl-PEG 2 kDa molar ratios (100:1, 5:1 and 1:1) show an increasing stabilizing effect with increasing molar ratios, the higher stabilizing effects being obtained for a molar ratio of 1:1. Further, the results show that there is a stabilizing effect occurring at very early stage of the mixture between the PEG derivative according to the invention and the protein and the formulations are stable over time (at least up to 72 hours).

### Example 3

### Synthesis of Trp-PEG Derivatives

The following PEG derivatives according to the invention (2 kDa and 5 kDa Trp-PEGs) (of Formula (II) wherein R³ is OR⁴ wherein R⁴ is substituted amide (e.g. formylamino-(1H-indo1-3-yl)-acetic acid); n is selected from 40-120; R¹ is optionally substituted C₁-C₆ alkyl (e.g. methyl)) were synthesized as depicted in Scheme 1 below:

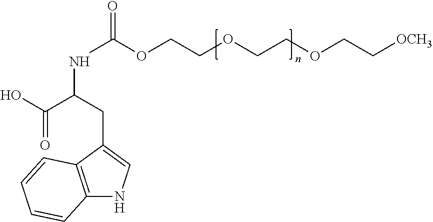

Synthesis of mPEG-p-nitrophenyl Carbonate 2 kDa (Method Adapted from U.S. Pat. No. 5,286,637)

1.76 mMol of dried mPEG-OH 2 kDa (Iris Biotech GmbH, Marktredwitz, Germany) were dissolved in anhydrous dichloromethane and 5.27 mMoles of p-nitrophenyl chloroformate (Acros Organics BVBA; Geels, Belgium) and 3.52 mMoles of dry triethyl amine were added (1:3:2 ratio). The pH was adapted between 7.5-8 and reaction was left to proceed at room temperature for 24 hours. Reaction was stopped by adding several drops of TFA until the solution was colourless, then dichloromethane was partially evaporated and precipitation from cold diethyl ether was performed. The solid collected via filtration was twice redissolved in dichloromethane, precipitated from cold diethyl ether, and collected via filtration. A slightly yellowish powder was obtained and dried under vacuum. ¹H-NMR (300 MHz, DMSO-d-6): 3.23 ppm, PEG CH₃—O-(s); 3.50 ppm, PEG —O—CH₂-(m); 7.55 ppm, p-nitrophenyl-aromate (d); 8.31 ppm, p-nitrophenyl-aromate (d). ¹³C-NMR (300 Mhz, DMSO-d-6): 58.06 ppm, PEG CH₃—O—; 69.52 ppm, PEG —O—CH₂—; 122.59 ppm, p-nitrophenyl-aromate; 125.34 ppm, p-nitrophenyl-aromate; 144.21 ppm, PEG —O—CH₂—C═O; 151.99 ppm, aromatic C₅H₄═C—NO₂; 155.27 ppm, PEG —CH₂—OCO—. FTIR: 3435; 2888; 2739; 2678; 2493; 1967; 1769; 1617; 1594; 1527; 1468; 1360; 1343; 1281; 1242; 1113; 1060; 963; 841; 663; 529 cm⁻¹. MS (MALDI-TOF): m/z 2201 (M⁺).

Synthesis of Tryptophan-mPEG 2 kDa (Method Adapted from U.S. Pat. No. 5,286,637)

0.018 Mol L-Tryptophan (Fluka (Sigma-Aldrich Chemie GmbH, Buchs, Switzerland)) were dissolved in anhydrous DMSO and pH was adapted to ˜8.3. Then, 1.76 mMol of dried mPEG-p-nitrophenyl carbonate 2 kDa obtained as described above were added. The pH was maintained at ˜8.3 and reaction was left to proceed at room temperature for 4 hours. Reaction was stopped by cooling to 0° C. and adapting pH to 3 with 2 M HCl. The aqueous phase was extracted with chloroform. The obtained organic phase was dried over anhydrous Na₂SO₄ and partially evaporated. Precipitation from cold diethyl ether was performed and the solid collected via filtration. The solid was once reprecipitated from cold diethyl ether, and twice from cold iso-propanol. A slightly yellowish powder was obtained and dried under vacuum. ¹H-NMR (300 MHz, DMSO-d-6): 3.17 ppm, Trp indole-CH₂—CH₂-(d); 3.24 ppm, PEG —CH₃—O-(s); 3.51 ppm, PEG —O—CH₂-(m); 4.17 ppm, Trp indole-CH₂—CH₂-(q); 6.98 ppm, Trp-indole (t); 7.06 ppm, Trp-indole (t); 7.16 ppm, Trp-indole (s); 7.32 ppm, Trp-indole (d); 7.51 ppm, Trp-indole (d); 10.82 ppm Trp-COOH (s). ¹³C-NMR (300 MHz, DMSO-d-6): 54.78 ppm, Trp indole-CH₂—CH₂—; 58.58 ppm, PEG CH₃—O—; 63.28 ppm, Trp indole-CH₂—CH₂—; 69.70 ppm, PEG —O—CH₂—; 110.02 ppm, Trp-indole; 111.33 ppm, Trp-indole; 117.79 ppm, Trp-indole; 120.80 ppm, Trp-indole; 123.65 ppm, Trp-indole; 126.88 ppm Trp-indole; 136.17 ppm, Trp-indole; 156.26 ppm, PEG —CH₂—OCO—NH—; 173.87 ppm, —COOH. FTIR: 3412; 2886; 2741; 2695; 2167; 1970; 1721; 1526; 1467; 1413; 1360; 1343; 1280; 1242; 1110; 963; 842; 745, 529 cm⁻¹. MS (MALDI-TOF): m/z 2266 (M⁺). [α]_{D}²⁰=−0.005.

Synthesis of mPEG-p-nitrophenyl Carbonate 5 kDa (Method Adapted from U.S. Pat. No. 5,286,637)

The reaction was performed as described for the mPEG-p-nitrophenyl carbonate 2 kDa, where 0.68 mMol of dried mPEG-OH 5 kDa, 2.03 mMoles of p-nitrophenyl chloroformate and 1.36 mMoles of dry triethyl amine were used. A slightly yellowish powder was obtained. ¹H-NMR (300 MHz, DMSO-d-6): 3.23 ppm, PEG CH₃—O-(s); 3.50 ppm, PEG -O—CH₂-(m); 7.55 ppm, p-nitrophenyl-aromate (d); 8.31 ppm, p-nitrophenyl-aromate (d). ¹³C-NMR (300 Mhz, DMSO-d-6): 58.27 ppm, PEG CH₃—O-; 69.70 ppm, PEG —O—CH₂—; 122.62 ppm, p-nitrophenyl-aromate; 125.33 ppm, p-nitrophenyl-aromate; 145.93 ppm, PEG —O—CH₂—C═O; 152.10 ppm, aromatic C₅H₄═C—NO₂; 154.76 ppm, PEG —CH₂—OCO—. FTIR: 3447; 2889; 2741; 2694; 2603; 2494; 1971; 1769; 1642; 1526; 1468; 1360; 1343; 1281; 1242; 1219; 1113; 1060; 963; 842; 529 cm⁻¹. MS (MALDI-TOF): m/z 4698 (M⁺).

Synthesis of Tryptophan-mPEG 5 kDa (Method Adapted from U.S. Pat. No. 5,286,637)

The reaction was performed as described for the Tryptophan-mPEG 2 kDa, where 0.68 mMol of dried mPEG-p-nitrophenyl carbonate 5 kDa (synthesized as described above) and 6.78 mMoles of L-Tryptophan were used. A slightly yellowish powder was obtained. ¹H-NMR (300 MHz, DMSO-d-6): 3.21 ppm, Trp indole-CH₂—CH₂-(d); 3.24 ppm, PEG —CH₃—O-s); 3.51 ppm, PEG —O—CH₂-(m); 4.18 ppm, Trp indole-CH₂—CH₂-(q); 6.96 ppm, Trp-indole (t); 7.02 ppm, Trp-indole (t); 7.14 ppm, Trp-indole (s); 7.32 ppm, Trp-indole (d); 7.51 ppm, Trp-indole (d); 10.81 ppm Trp —COOH (s). ¹³C-NMR (300 MHz, DMSO-d-6): 54.85 ppm, Trp indole-CH₂—CH₂—; 58.04 ppm, PEG CH₃—O—; 63.46 ppm, Trp indole-CH₂—CH₂—; 69.72 ppm, PEG —O—CH₂—; 110.83 ppm, Trp-indole; 111.34 ppm, Trp-indole; 117.99 ppm, Trp-indole; 120.90 ppm, Trp-indole; 124.11 ppm, Trp-indole; 127.04 ppm Trp-indole; 136.62 ppm, Trp-indole; 156.24 ppm, PEG —CH₂—OCO—NH—; 173.68 ppm, —COOH. FTIR: 3438; 2885; 2741; 2695; 1969; 1719; 1647; 1467; 1360; 1343; 1281; 1242; 1112; 1060; 963; 842; 746; 529 cm⁻¹. MS (MALDI-TOF): m/z 4772 (M⁺). [α]_{D}²⁰=−0.002.

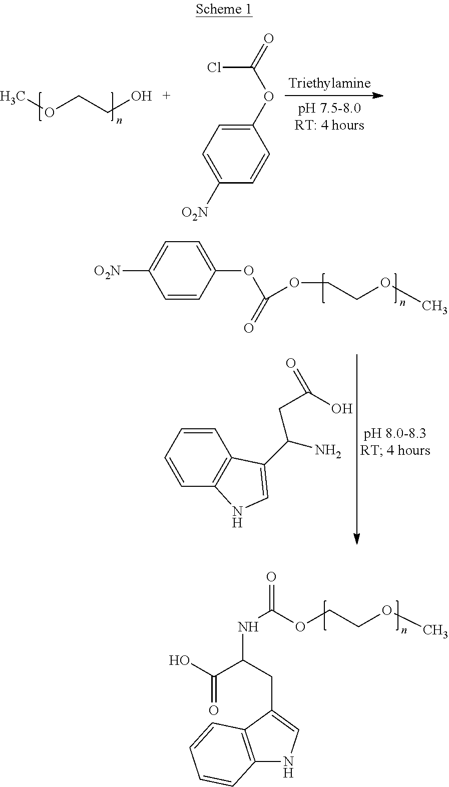

### Example 4

### Synthesis of phenylbutylamine-PEG Derivative

The following PEG derivative according to the invention (2 kDa phenylbutylamine-PEG) (of Formula (II) wherein R³ is OR⁴ wherein R⁴ is substituted amide (e.g. N-(4-phenyl-butyl)-formamide); n is selected from 40-50; R¹ is optionally substituted C₁-C₆ alkyl (e.g. methyl)) was synthesized as follows:

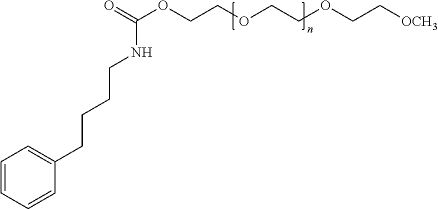

Synthesis of phenylbutylamine-mPEG 2 kDa

0.069 Mol phenylbutylamine (Sigma-Aldrich Chemie GmbH, Buchs, Switzerland) were dissolved in anhydrous dichloromethane 1.39 mMol of dried mPEG-p-nitrophenyl carbonate 2 kDa (synthesized as described in Example 3) were added. The pH was maintained at ˜10.4 and reaction was left to proceed at room temperature for 6 hours. Reaction was stopped by evaporation of dichloromethane. The residue was redissolved in 2 M HCl and pH was adapted to 2. The aqueous phase was extracted with dichloromethane. The obtained organic phase was dried over anhydrous Na₂SO₄ and partially evaporated. Precipitation from cold diethyl ether was performed and the solid collected via filtration. The solid was once reprecipitated from cold diethyl ether, and once from cold iso-propanol. A white powder was obtained, dried under vacuum and redissolved in milliQ™ water. The solution was filtered through a 0.22 μm Millex-GV™ filter (Millipore, Carrigtwohil, Co. Cork, Ireland) and freeze dried (Freeze dryer Micro Modulyo™, Edwards High Vacuum Int., Crawley Sussex, UK). ¹H-NMR (300 MHz, DMSO-d-6): 1.40 ppm phenylbutylamine —CH₂—; 1.54 ppm phenylbutylamine —CH₂—; 3.24 ppm, PEG —CH₃—O—; 3.51 ppm, PEG —O—CH₂—; 7.19 ppm phenylbutylamine Ar. ¹³C-NMR (300 MHz, DMSO-d-6): 28.02 ppm phenylbutylamine —CH₂—; 29.07 ppm phenylbutylamine —CH₂—; 34.66 ppm phenylbutylamine —CH₂—; 58.10 ppm, PEG CH₃—O—; 69.50 ppm, PEG —O—CH₂—; 125.49 ppm phenylbutylamine Ar; 128.40 ppm phenylbutylamine Ar; 141.87 ppm phenylbutylamine Ar; 155.76 ppm phenylbutylamine Ar. FTIR: 2883; 1964; 1719; 1537; 1466; 1359; 1341; 1279; 1240; 1146; 1098; 1059; 959; 841; 749; 700. MS (MALDI-TOF): m/z 2124 (M⁺).

### Example 5

### Synthesis of Cholesterol-PEG Derivatives

The following PEG derivatives according to the invention (2 kDa and 5 kDa Cholesterol-PEGs) (of Formula (II) wherein R³ is OR⁴ wherein R⁴ is substituted heteroaryl (e.g. 3-(1,5-Dimethyl-hexyl)-3a,6,6-trimethyl-2,3,3a,4,5,5a,6,9,9a,9b-decahydro-1H-cyclopenta[a] naphthalene), n is selected from 40-120; R¹ is H) were purchased to NOF Corporation, Tokyo, Japan (Sunbright CS-020 and -050).

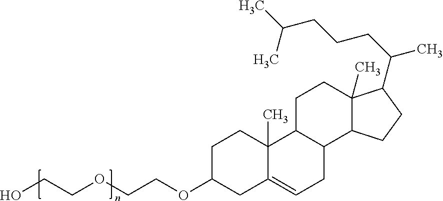

### Cholesteryl-PEG 2 kDa

¹H-NMR (300 MHz, DMSO-d-6): 0.69 ppm; 0.87 ppm; 0.89 ppm; 0.98 ppm; 1.14 ppm; 2.34 ppm,; 3.14 ppm; 3.55 ppm; 5.34 ppm. ¹³C-NMR (300 MHz, DMSO-d-6): 11.61 ppm; 18.48 ppm; 19.00 ppm; 20.56 ppm; 22.34 ppm; 22.61 ppm; 23.15 ppm; 23.81 ppm; 27.35 ppm; 27.74 ppm; 27.98 ppm; 31.35 ppm; 35.19 ppm; 35.60 ppm; 36.23 ppm; 36.64 ppm; 41.79 ppm; 49.55 ppm; 55.52 ppm; 56.13 ppm; 60.13 ppm; 66.56 ppm; 69.72 ppm; 72.28 ppm; 78.36 ppm; 99.56 ppm; 120.97 ppm; 140.44 ppm.

FTIR: 2883; 1967; 1466; 1359; 1341; 1279; 1240; 1146; 1102; 1060; 958; 841; 735. MS (MALDI-TOF): m/z 1994 (M+).

### Cholesteryl-PEG 5 kDa

¹H-NMR (300 MHz, DMSO-d-6): 0.68 ppm; 0.84 ppm; 0.88 ppm; 0.97 ppm; 1.11 ppm; 2.33 ppm; 3.13 ppm; 3.54 ppm; 5.34 ppm. ¹³C-NMR (300 MHz, DMSO-d-6): 11.61 ppm; 18.48 ppm; 19.00 ppm; 20.54 ppm; 22.34 ppm; 22.62 ppm; 23.13 ppm; 23.81 ppm; 27.34 ppm; 27.74 ppm; 27.99 ppm; 31.36 ppm; 35.15 ppm; 35.59 ppm; 36.64 ppm; 41.78 ppm; 49.54 ppm; 55.52 ppm; 56.13 ppm; 60.13 ppm; 66.57 ppm; 69.71 ppm; 72.28 ppm; 78.34 ppm; 121.01 ppm; 140.39 ppm.

FTIR: 2882; 2740; 1969; 1466; 1359; 1340; 1279; 1240; 1145; 1102; 1059; 957; 841; 735. MS (MALDI-TOF): m/z 4858 (M+).

### Example 6

### Comparison of the Aggregation Propensity of Calcitonin or Hen Egg White Lysozyme Alone and in Combination with Further PEG Derivatives

In order to assess the stabilizing effect of PEG derivatives according to the invention, the aggregation propensity of salmon calcitonin (sCT) or hens egg white lysozyme (HEWL) is assayed in presence or absence of PEG derivatives according to the invention.

Hen egg white lysozyme is a 130-amino acid polypeptide of 14.4 kDa (EC 3.2.1.17, Jolles, 1969, Angewandte Chemie, International Edition, 8, 227-239) which can be separated by high-speed countercurrent chromatography using a reverse micellar system as described in Xue-li Cao et al., 2007, Journal of Liquid Chromatography & Related Technologies, 30(17), 2593-2603. It presents bacteriolytic and immunological modulating properties (Mine et al., 2004, J. Agric. Food Chem., 52:1088-1094; Eun-Ha Kim et al., 2002, Immunopharmacology and Immunotoxicology, 24(3), pp. 423-440). Aggregation studies of sCT were performed as described in Example 2. Aggregation studies of HEWL were performed as follows: HEWL (Sigma-Aldrich Chemie GmbH, Buchs, Switzerland) in a final concentration of 2.1 mM per well with and without the respective excipients to be tested, and non-conjugated PEG-derivatives were prepared in 50 mM sodium phosphate buffer pH 12.2. Aggregation is followed in UV-transparent 384-well Costar® plates as described in Example 2. The protein formulations according to the invention in Table 4 below were tested:

TABLE 4

| | | Molar ratios |
|---|---|---|
| | | Protein/PEG |
| Protein | PEG-derivatives | Derivative |
| sCT | Trp-PEG 2 kDa | 2:1; 1:1; 1:2; 1:5; 1:10 |
| sCT | Trp-PEG 5 kDa | 1:1; 1:5 |
| HEWL | phenylbutyl amine PEG 2 kDa | 1:1; 1:10 |
| HEWL | Cholesterol-PEG 2 kDa | 1:1 |
| HEWL | Cholesterol-PEG 5 kDa | 1:1 |

Aggregation Propensity of Proteins Alone and in Combination with PEG Derivatives

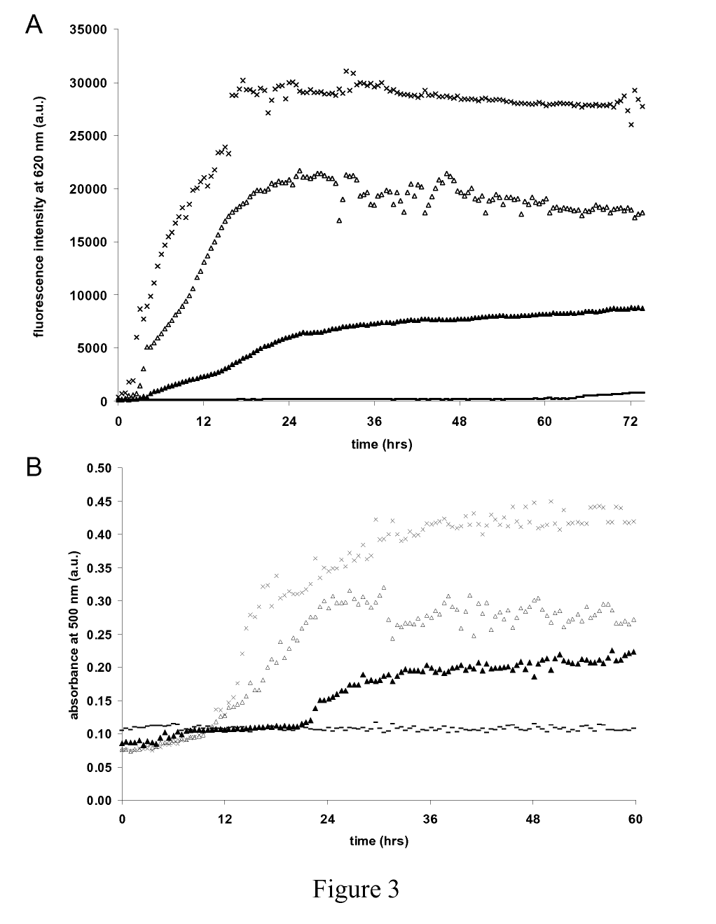

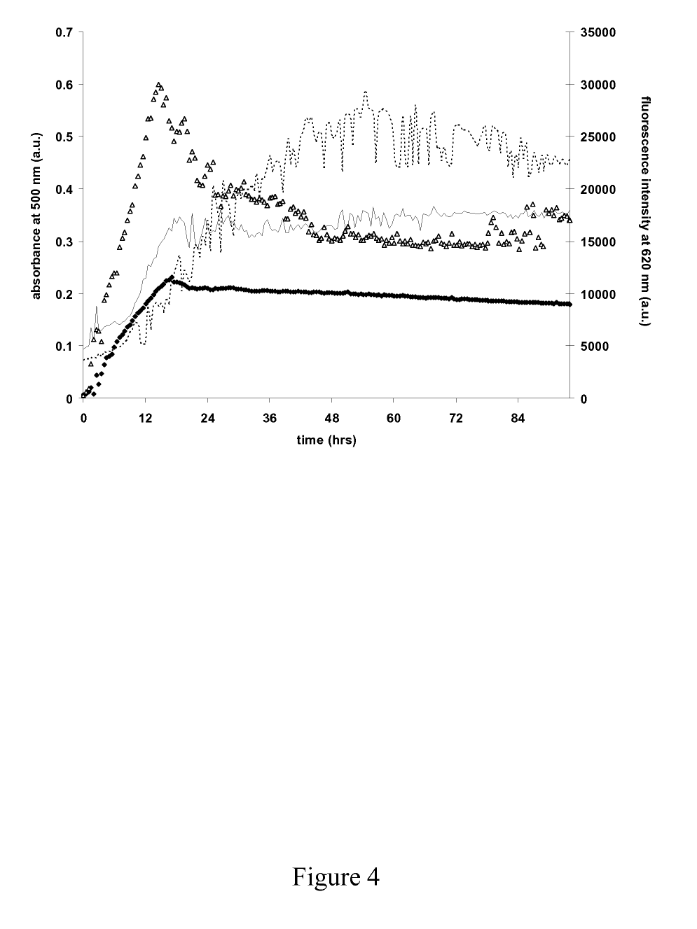

FIGS. 3A and 3B show that with increasing amounts of Trp-mPEG 2 kDa added, the lag phase of aggregation was prolonged and the aggregation of sCT was reduced. Reduced turbidity and Nile Red fluorescence intensity were observed in the following order: i) sCT, ii) sCT:Trp-mPEG 2 kDa molar ratio=1:1, and iii) sCT:Trp-mPEG 2 kDa molar ratio=1:5, iv) sCT:Trp-mPEG 2 kDa molar ratio=1:10, demonstrating a reduction of sCT aggregation. For sCT:Trp-mPEG 2 kDa molar ratio=1:10 the aggregation was completely suppressed up to 64 hours. FIG. 4 shows that the aggregation of sCT in 10 mM sodium citrate buffer pH 6 was also reduced in presence of Trp-mPEG 5 kDa in a molar ratio sCT:Trp-mPEG 5 kDa of 1:5.

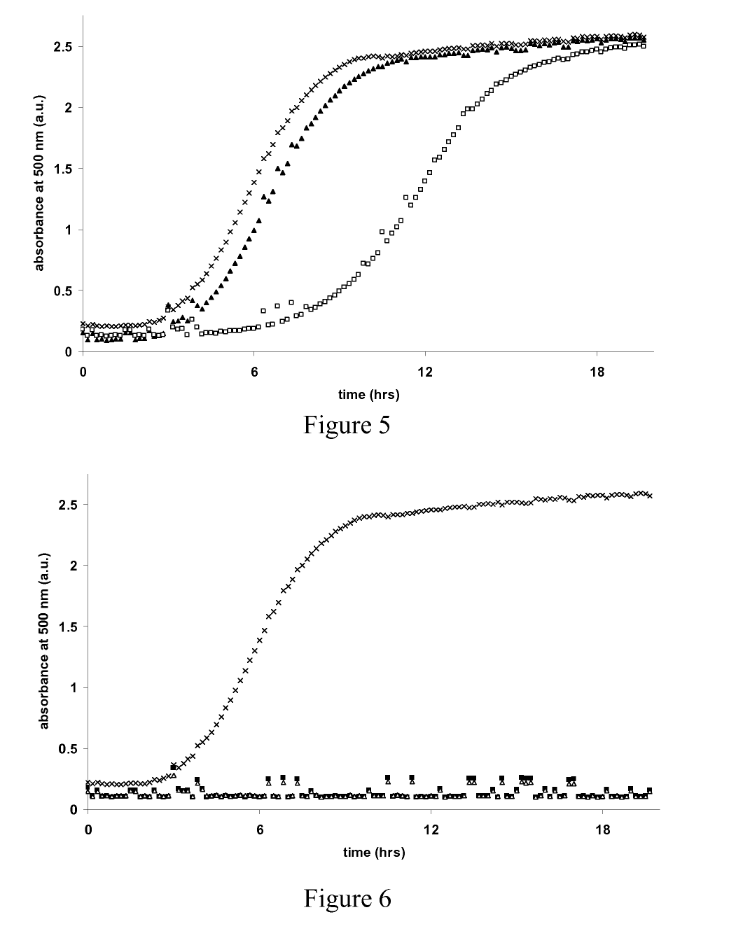

With increasing concentration of phenylbutylamine-mPEG 2 kDa a prolongation in the onset of HEWL aggregation was observed (FIG. 5). Cholesterol-PEGs of 2 and 5 kDa completely suppressed the aggregation of HEWL (FIG. 6) in a molar ratio protein:PEG derivative of 1:1.

### Example 7

### Comparison of the Stability of Sterile Solution for Injection of Calcitonin Alone and in Combination with PEG Derivatives

Stability of formulations according to the invention is compared to the stability of a sterile solution for injection containing 0.033 mg/ml (resp. 200 I.U.) of sCT (Miacalcin®, Novartis, Switzerland) which compositions are described under Table 5 below. The formulations from Table 5 below are prepared as follows: first a solution of the respective amounts of acetic acid, phenol, sodium acetate trihydrate, and sodium chloride in a fraction of water for injection (less than 1 ml) are prepared. In the case of formulations containing DNS-mPEG or Trp-mPEG 2 kDa, the respective amounts of the PEG derivatives are added to and dissolved in the solution prepared in the first step. Then, sCT is added and dissolved. Finally, the volume is completed with water for injection to 1 ml.

TABLE 5

| | | sCT:D-PEG2 | sCT:T-PEG2 | sCT:T-PEG2 | Control | Control | Control |
|---|---|---|---|---|---|---|---|
| Composition | Miacalcin ® | 1:1 | 1:1 | 1:10 | D-PEG2 | T-PEG2 | T-PEG2 |
| sCT (mg) | 0.033 | 0.033 | 0.033 | 0.033 | — | — | — |
| acetic acid | 2.25 | 2.25 | 2.25 | 2.25 | 2.25 | 2.25 | 2.25 |
| (mg) | | | | | | | |
| phenol (mg) | 5.0 | 5.0 | 5.0 | 5.0 | 5.0 | 5.0 | 5.0 |
| sodium | 2.0 | 2.0 | 2.0 | 2.0 | 2.0 | 2.0 | 2.0 |
| acetate | | | | | | | |
| trihydrate | | | | | | | |
| (mg) | | | | | | | |
| sodium | 7.5 | 7.5 | 7.5 | 7.5 | 7.5 | 7.5 | 7.5 |
| chloride | | | | | | | |
| (mg) | | | | | | | |
| water for | qs to 1 ml | qs to 1 ml | qs to 1 ml | qs to 1 ml | qs to 1 ml | qs to 1 ml | qs to 1 ml |
| injection | | | | | | | |
| PEG- | — | 0.021 | 0.021 | 0.21 | 0.021 | 0.021 | 0.21 |
| derivative | | | | | | | |
| (mg) | | | | | | | |

D-PEG2 = DNS-PEG 2 kDa;
T-PEG2 = Trp-PEG 2 kDa

All formulations are prepared in glass vials protected from light and stressed by two methods, i) horizontal shaking at room temperature (25° C.) and by ii) storage at 37° C. At preselected time points (e.g. bi-weekly), one or more of the following measurements is performed:
UV absorbance scan (230-550 nm), Nile red fluorescence emission spectra and the intrinsic fluorescence emission spectra of the dansyl- or Trp-headgoup is measured by a microplate reader on a 96-well plate as described above. The intrinsic tyrosine emission of sCT is measured with samples containing the dansyl-PEGs to follow conformational changes.
Intrinsic fluorescence emission/excitation spectra of the dansyl- or Trp-headgoup, intrinsic tyrosine fluorescence emission/excitation of sCT, 90° light scatter, anisotropy, UV absorbance spectra is measured. Furthermore, Nile Red fluorescence emission/excitation, 90° light scatter, anisotropy are measured. Brightfield and Nile Red fluorescence microscopy are performed. All measurements are performed at various settings.

The extent of aggregation of sCT is used as a measure of the stabilizing effect of the PEG derivatives according to the invention as compared to a commercial formulation of this protein.

### Example 8

### Pharmacokinetic Studies

In order to assess the stabilizing effect of PEG derivatives according to the invention, the bioavailability of a protein is assayed in presence or absence of PEG derivatives according to the invention. The stabilized protein formulation is injected i.v. and s.c. in suitable animals (mice, rats, rabbits). Blood samples are drawn at pre-determined intervals and subjected to treatment allowing quantitative measurement of protein concentration by standard assay (e.g., ELISA). Protein solution in the absence of stabilizing PEG-derivative serves as control. Pharmacokinetic parameters, including tₘₐₓ, cₘₐₓ, AUC, t_{1/2}, and kₑₗ is determined for the stabilized protein and the control group and for both application routes and compared to each other.

### Example 9

### Immunogenicity Studies

In order to assess the stabilizing effect of PEG derivatives according to the invention, the immunogenicity of a protein is assayed in presence or absence of PEG derivatives according to the invention. Detection and characterization of binding antibodies (BABs) is performed by solid phase binding immunoassay, e.g., enzyme-linked immunosorbent assay (ELISA), preferably in bridging mode using labeled protein for detection of BABs. Specificity of the detected antibodies is assessed by immunoblotting, while their neutralizing activity is determined by specific bioassay measuring the bioactivity of the protein.

1-27. (canceled)

28. A stable protein formulation, said formulation comprising a non-covalent combination of an aqueous carrier, a protein and a PEG derivative, wherein the PEG derivative comprises at least one polyethylene glycol moiety covalently grafted to a hydrophobic group

29. The formulation according to claim 28, wherein the formulation is a pharmaceutical formulation.

30. The formulation according to claim 28, wherein the protein is at a concentration from about 0.01 ng/ml to about 500 mg/ml.

31. The formulation according to claim 28, wherein the PEG derivative is at a concentration from about 0.001 ng/ml to 1 g/ml.

32. The formulation according to claim 28, wherein the PEG derivative is an mPEG derivative.

33. The formulation according to claim 28, further comprising an excipient.

34. The formulation according to claim 28, wherein the hydrophobic group is selected from dansylamide, phenylbutylamine, cholesterol and an amino acid.

35. The formulation according to claim 28, wherein the hydrophobic group is a benzyl group.

36. The formulation according to claim 28, wherein the PEG derivative is of Formula (II): R¹—(OCH₂CH₂)ₙ—R³, wherein R³ is selected from OR⁴, wherein R⁴ is selected from substituted heteroaryl, substituted amide or substituted amine; n is selected from 40-120; and R¹ is selected from H and optionally substituted C₁-C₆ alkyl.

37. The formulation according to claim 28, wherein the PEG derivative is selected from: 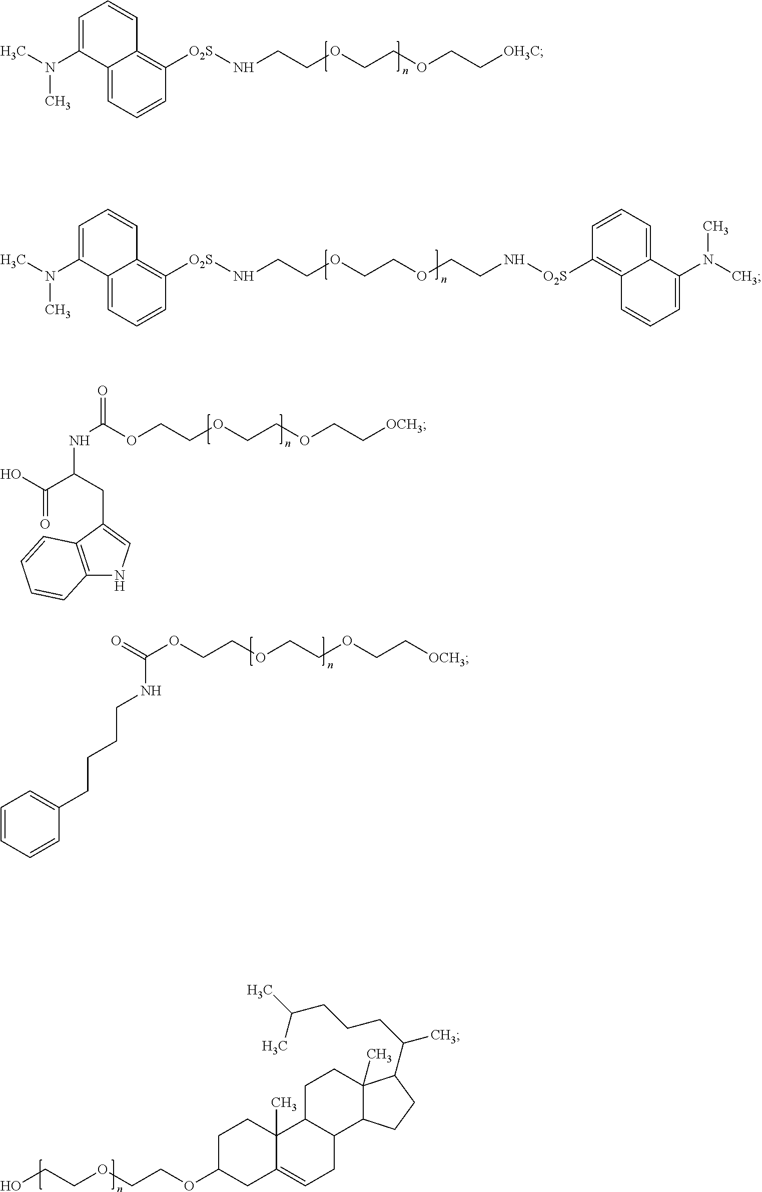
and pharmaceutically acceptable salts, pharmaceutically acceptable derivatives or isomers thereof.

38. The formulation according to claim 28, wherein the protein is selected from salmon calcitonin (sCT) and hen egg white lysozyme (HEWL).

39. The formulation according to claim 28, wherein the molar ratio PEG derivative to protein is 1:1.

40. A method of stabilizing a protein in aqueous solution by non-covalently combining said protein with a PEG derivative, wherein the PEG derivative comprises at least one polyethylene glycol moiety covalently grafted to a hydrophobic group.

41. The method according to claim 40, wherein the PEG derivative is an mPEG derivative.

42. The method according to claim 40, wherein the hydrophobic group is selected from dansylamide, phenylbutylamine, cholesterol and an amino acid.

43. The method according to claim 40, wherein the PEG derivative is of Formula (II): R¹—(OCH₂CH₂)ₙ—R³, wherein R³ is selected from OR⁴, wherein R⁴ is selected from substituted heteroaryl, substituted amide and substituted amine; n is selected from 40-120; and R¹ is selected from H and optionally substituted C₁-C₆ alkyl.

44. The method according to claim 40, wherein the PEG derivative is selected from: 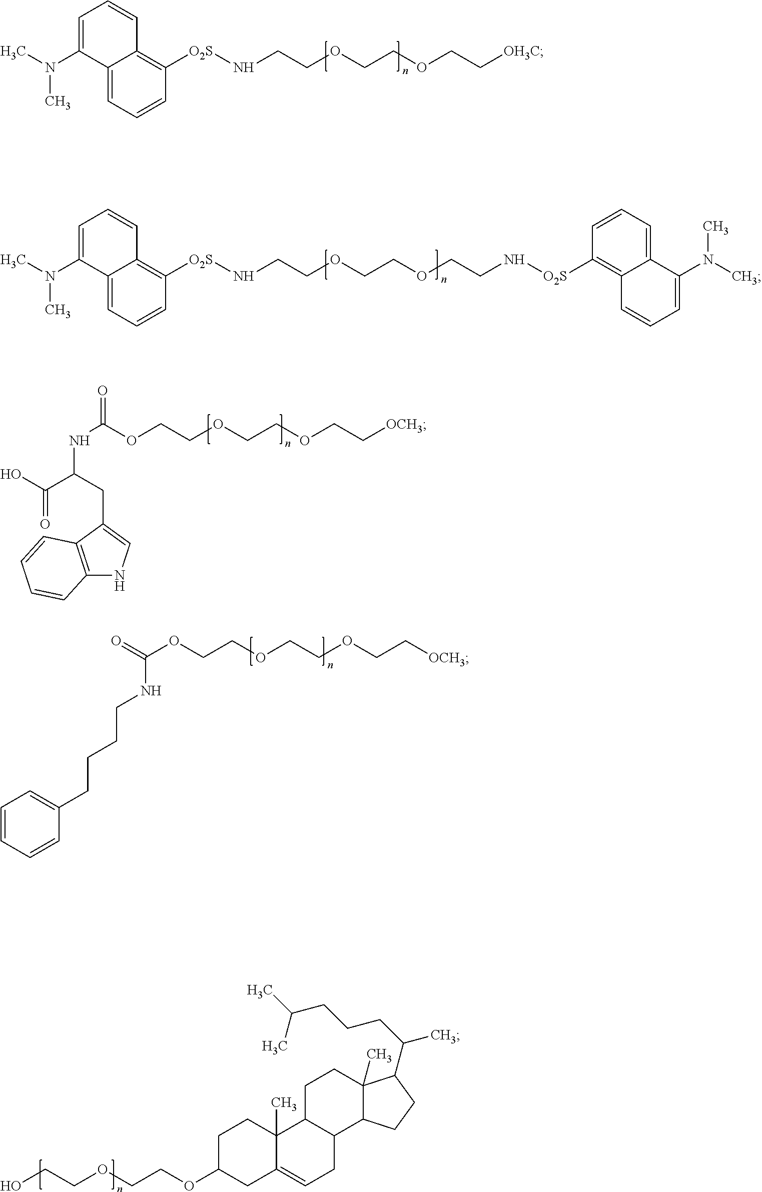
and pharmaceutically acceptable salts, pharmaceutically acceptable derivatives or isomers thereof.

45. A process for the preparation of a protein or a formulation thereof comprising the steps of:
(i) non-covalently combining a protein with a PEG derivative into a liquid mixture or forming said protein in a liquid medium containing a PEG derivative, wherein the PEG derivative comprises at least one polyethylene glycol moiety covalently grafted to a hydrophobic group; and
(ii) collecting the liquid mixture or liquid medium obtained under step (i) containing the stabilized non-covalent protein thereof wherein the percentage of monomers of protein is increased as compared to protein prepared in absence of the said PEG derivative.

46. The process according to claim 45, wherein the PEG derivative is an mPEG derivative.

47. The process according to claim 45, wherein the hydrophobic group is selected from dansylamide, phenylbutylamine, cholesterol and an amino acid.

48. The process according to claim 45, wherein the PEG derivative is of Formula (II): R¹—(OCH₂CH₂)ₙ—R³, wherein R³ is selected from OR⁴, wherein R⁴ is selected from substituted heteroaryl, substituted amide and substituted amine; n is selected from 40-120; and R¹ is selected from H and optionally substituted C₁-C₆ alkyl.

49. The process according to claim 45, wherein the PEG derivative is selected from: 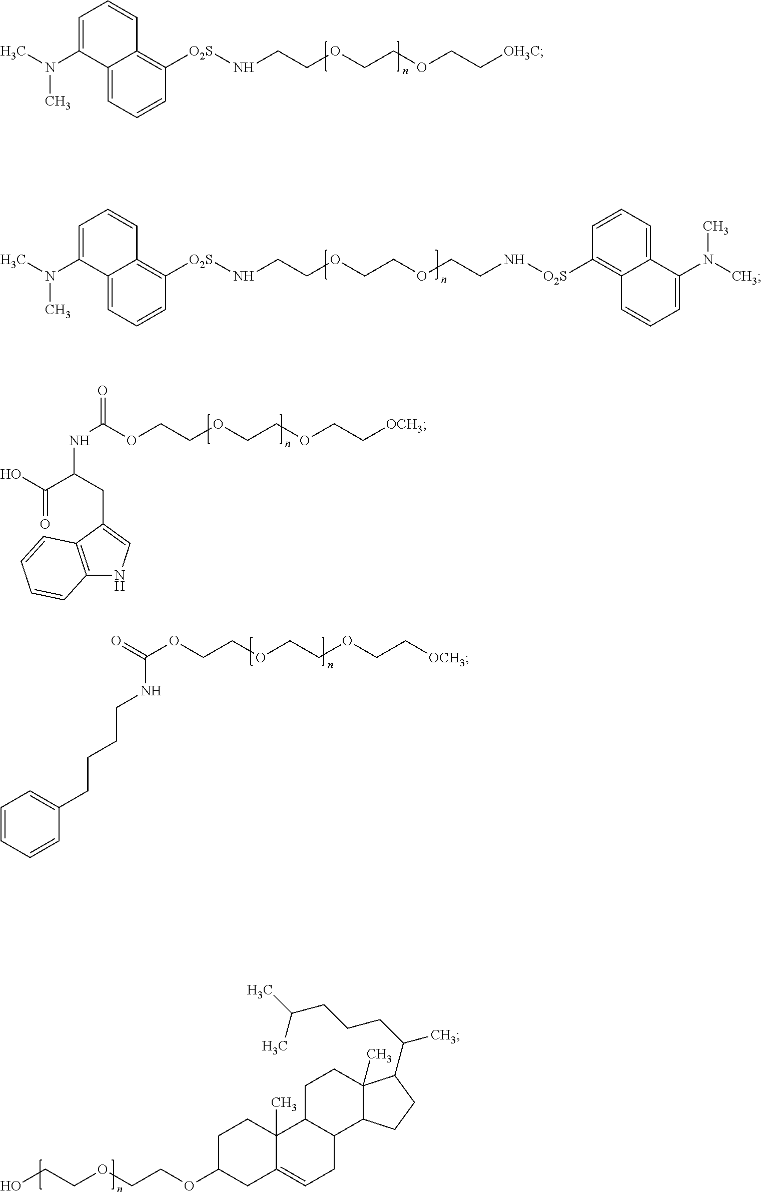
and pharmaceutically acceptable salts, pharmaceutically acceptable derivatives or isomers thereof.

50. A PEG derivative comprising at least one polyethylene glycol moiety covalently grafted to a hydrophobic group, wherein the hydrophobic group is selected from dansylamide, tryptophan, phenylbutylamine, cholesterol, and an amphipathic peptide.

51. The PEG derivative according to claim 50, said PEG derivative having the formula: 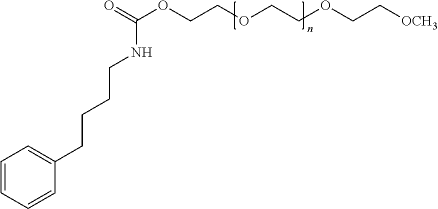
and pharmaceutically acceptable salts, pharmaceutically acceptable derivatives or isomers thereof.

52. A method of making a pharmaceutical composition comprising combining a PEG derivative according to claim 50 with a pharmaceutically acceptable carrier.

53. A process for the preparation of a PEG derivative comprising reacting an mPEG-p-nitrophenyl carbonate with phenylbutylamine in an anhydrous solvent at a pH between about 9 and 11 at room temperature.

54. A kit for reconstituting a protein in solution comprising in one container a lyophilized protein, and a PEG derivative in another container or another part of said container, optionally together with a container containing a sterile buffer for reconstituting the protein and optionally with instruction for use of said kit, wherein the PEG derivative comprises at least one polyethylene glycol moiety covalently grafted to a hydrophobic group.
